SEQUENCE LISTING (32 sequences)

SEQ ID NO: 1 - protein, 360 aa, Homo sapiens
GAGGTGCAGCTGTTGGAGTCTGGGGGAGGCTTGGTACAGCCTGGGGGGTCCCTGAGACTCTCCTGTGCAGCCTCTGGATTCACCTTTAGCAGCTATGCCATGAGCTGGGTCCGCCAGGCTCCAGGGAAGGGGCTGGAGTGGGTCTCAGCTATTAGTGGTAGTGGTGGTAGCACATACTACGCAGACTCCGTGAAGGGCCGGTTCACCATCTCCAGAGACAATTCCAAGAACACGCTGTATCTGCAAATGAACAGCCTGAGAGCCGAGGACACGGCCGTGTATTACTGTGCGAGAGGTCGGACTGAGGTGGCAACCCCCGGCGCCTACTGGGGCCAAGGGACAATGGTCACCGTCTCGAGT

SEQ ID NO: 2 - protein, 120 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGRTEVATPGAYWGQGTMVTVSS

SEQ ID NO: 3 - DNA, 333 bp, Homo sapiens
caggctgtgctgactcagccgtcctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccgtcactgggctccaggctgaggatgaggctgattattactgccagtcctatgacagcagcctgagtgatgtggtattcggcggagggaccaagctgaccgtccta

SEQ ID NO: 4 - protein, 111 aa, Homo sapiens
QAVLTQPSSVSGAPGQRVTISCTGSSSNIGAGYDVHWYQQLPGTAPKLLIYGNSNRPSGVPDRFSGSKSGTSASLAVTGLQAEDEADYYCQSYDSSLSDVVFGGGTKLTVL

SEQ ID NO: 5 - DNA, 366 bp, Homo sapiens
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagatatcgggactatggtggtaactcccacctctttgactactgggggcaagggaccacggtcaccgtctcgagt

SEQ ID NO: 6 - protein, 122 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARYRDYGGNSHLFDYWGQGTTVTVSS

SEQ ID NO: 7 - DNA, 321 bp, Homo sapiens
gaaattgtgatgacgcagtctccgtcctccctgcctgcctctgtaggagacagagtcaccatcacttgtcgggcaagtcagaacattaagacctatttgcactggtaccaacagaagccagggaaagcccctaacctcctgatctatgctgcatccaatttgcaaattggggtcccatcaaggttcagtggcagtggatctgggacagattttactctcaccatcagcagtctgcaacctgaagattttgctacttacttctgtcaacagagttacattacccctcccaccttcggccaagggacacgactggagattaaa

SEQ ID NO: 8 - protein, 107 aa, Homo sapiens
EIVMTQSPSSLPASVGDRVTITCRASQNIKTYLHWYQQKPGKAPNLLIYAASNLQIGVPSRFSGSGSGTDFTLTISSLQPEDFATYFCQQSYITPPTFGQGTRLEIK

SEQ ID NO: 9 - DNA, 354 bp, Homo sapiens
ggggtccagctggtacagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtatattactgtgctagaagggttatttcgggtgcttttgatatctggggccaggggacaatggtcaccgtctcgagt

SEQ ID NO: 10 - protein, 118 aa, Homo sapiens
GVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARRVISGAFDIWGQGTMVTVSS

SEQ ID NO: 11 - DNA, 321 bp, Homo sapiens
gacatccagatgacccagtctccttccaccctgtctgcatctattggagacagagtcaccatcacctgccgggccagtgagggtatttatcactggttggcctggtatcagcagaagccagggaaagcccctaaactcctgatctataaggcctctagtttagccagtggggccccatcaaggttcagcggcagtgggtctgggacagatttcactctcaccatcagcagcctgcagcctgatgattttgcaacttattactgccaacaatatagtaattatccgctcactttcggcggagggaccaagctggagatcaaa

SEQ ID NO: 12 - protein, 107 aa, Homo sapiens
DIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIK

SEQ ID NO: 13 - DNA, 369 bp, Homo sapiens
caggtgcagctggtgcagtctggggctgaggtgaagaagcctggggcctcagtgaaggtttcctgcaaggcatctggatacaccttcaccagctactatatgcactgggtgcgacaggcccctggacaggggcttgagtggatggggataatcaaccctagtgatggtagcacaaggtacgtagagaagttccagggcagagtcaccatgaccagggacacgtccacgagcacagtctacatggagttgagcagcctgagatctgaggacacggccgtgtatttctgtgcgagaggcatgggacccggcccccactaccacttctacatggacgtctggggcaaagggacaatggtcaccgtctcctca

SEQ ID NO: 14 - protein, 123 aa, Homo sapiens
QVQLVQSGAEVKKPGASVKVSCKASGYTFTSYYMHWVRQAPGQGLEWMGIINPSDGSTRYVEKFQGRVTMTRDTSTSTVYMELSSLRSEDTAVYFCARGMGPGPHYHFYMDVWGKGTMVTVSS

SEQ ID NO: 15 - DNA, 324 bp, Homo sapiens
tcgtctgagctgactcaggaccctgctgtgtctgtggccttgggacagacagtcaggatcacttgccaaggagacagtctcagaagctattacacaaactggttccagcagaagccaggacaggcccctctacttgtcgtctatgctaaaaataagcggccctcagggatcccagaccgattctctggctccagctcgggaaacacagcttccttgaccatcactggggctcaggcggaagatgaggctgactattactgtcattcccgggacagcggtggtaaccatgtgcttttcggcggagggaccaagctgaccgtccta

SEQ ID NO: 16 - protein, 108 aa, Homo sapiens
SSELTQDPAVSVALGQTVRITCQGDSLRSYYTNWFQQKPGQAPLLVVYAKNKRPSGIPDRFSGSSSGNTASLTITGAQAEDEADYYCHSRDSGGNHVLFGGGTKLTVL

SEQ ID NO: 17 - DNA, 375 bp, Homo sapiens
caggtgcagctggtgcagtctggggctgaggtgaagaagcctggggcctcagtgaaggtctcctgcaaggcttctggttacacctttaccggccagtacatccactgggtgcgacaggcccctggacaagggcttgagtggatgggatggatcagcgcttacaatggttacacagactatgcacagaaggtccagggcagagtcaccatgaccacagacacatccaccagcacagcctacatggagctgaggagcctgagatctgacgacacggccgtgtattactgtgcgagagaggtgtggccagtggcagcagctgatacattcagtgtttttgatatctggggccgaggaaccctggtcaccgtctcgagt

SEQ ID NO: 18 - protein, 125 aa, Homo sapiens
QVQLVQSGAEVKKPGASVKVSCKASGYTFTGQYIHWVRQAPGQGLEWMGWISAYNGYTDYAQKVQGRVTMTTDTSTSTAYMELRSLRSDDTAVYYCAREVWPVAAADTFSVFDIWGRGTLVTVSS

SEQ ID NO: 19 - DNA, 324 bp, Homo sapiens
tcgtctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatgtggtattcggcggagggaccaagctgaccgtccta

SEQ ID NO: 20 - protein, 108 aa, Homo sapiens
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 21 - DNA, 354 bp, Homo sapiens
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagagatgggaccacggggttgcatgactcctggggccaagggacaatggtcaccgtctcgagt

SEQ ID NO: 22 - protein, 118 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDGTTGLHDSWGQGTMVTVSS

SEQ ID NO: 23 - DNA, 333 bp, Homo sapiens
cagtctgtgttgacgcagccgccctcagcgtctgggacccccggacagagggtcactatctcttgttctggaagcagttccaacgtcggaagtaattttgtatattggtaccagcagttcccaggaacggcccccaaactcctcatctataggaataatcagcggccctcaggggtccctgaccgattctctggctccaagtccggcacctcagcctccctggccattagtggcctccggtccgaggatgaggctgattattactgtgcagcatgggatgacaccctgaatggtcactacgtgttcggcggagggaccaagctgaccgtccta

SEQ ID NO: 24 - protein, 111 aa, Homo sapiens
QSVLTQPPSASGTPGQRVTISCSGSSSNVGSNFVYWYQQFPGTAPKLLIYRNNQRPSGVPDRFSGSKSGTSASLAISGLRSEDEADYYCAAWDDTLNGHYVFGGGTKLTVL

SEQ ID NO: 25 - DNA, 354 bp, Homo sapiens
gaggtccagctggtgcagtctggagcagaggtgaaaaagcccggggagtctctgaagatctcctgtaagggatatggatacgatttcagtcgcgactggatcgcctgggtgcgccagatgcccgggaaaggcctggagtggatggggatcatctatcctggtgactctgataccagatacagcccgtccttcgaaggccaggtcaccatctcagccgacaagtccatcagcaccgcctacctgcagtggagaagcctgaaggcctcggacaccgccatgtattactgtgcgagacaacggaggttggggtggttcgacccctggggccaggggacaatggtcaccgtctcttca

SEQ ID NO: 26 - protein, 118 aa, Homo sapiens
EVQLVQSGAEVKKPGESLKISCKGYGYDFSRDWIAWVRQMPGKGLEWMGIIYPGDSDTRYSPSFEGQVTISADKSISTAYLQWRSLKASDTAMYYCARQRRLGWFDPWGQGTMVTVSS

SEQ ID NO: 27 - DNA, 330 bp, Homo sapiens
cggtctgtgttgacgcagccgccctcagtgtctgcggccccaggacagaaggtcaccatttcctgctctggaagcacctccaacattgggaataattatgtctcctggtaccaacagcacccaggcaaagcccccaaactcatgatttatgatgtcagtaagcggccctcaggggtccctgaccgattctctggctccaagtctggcaactcagcctccctggacatcagtgggctccagtctgaggatgaggctgattattactgtgcagcatgggatgacagcctgagtgaatttctcttcggaactgggaccaagctgaccgtccta

SEQ ID NO: 28 - protein, 110 aa, Homo sapiens
RSVLTQPPSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVL

SEQ ID NO: 29 - DNA, 369 bp, Homo sapiens
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagacatttaccgtctgggtctagcagcagttgggcctttgactcctgggggcgagggaccacggtcaccgtctcgagt

SEQ ID NO: 30 - protein, 123 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARHLPSGSSSSWAFDSWGRGTTVTVSS

SEQ ID NO: 31 - DNA, 330 bp, Homo sapiens
tcctatgtgctgactcagccaccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccagtctgaggatgaggctgattattactgtgaggcatgggatgacaacgtcgatggtccggtgttcggcggggggaccaagctgaccgtccta

SEQ ID NO: 32 - protein, 110 aa, Homo sapiens
SYVLTQPPSASGTPGQRVTISCSGSSSNIGSNYVYWYQQLPGTAPKLLIYRNNQRPSGVPDRFSGSKSGTSASLAISGLQSEDEADYYCEAWDDNVDGPVFGGGTKLTVL

(19) United States
(12) Patent Application Publication
(10) Pub. No.: US 2009/0263383 A1
(43) Pub. Date: Oct. 22, 2009

(54) Antibodies to angiogenesis inhibiting domains of CD148

(75) Inventors: James F. Smothers, Quincy, MA (US); William C. Fanslow, III, Normandy Park, WA (US); Revital Kariv, Bellevue, WA (US)

Correspondence Address: AMGEN INC.;LAW DEPARTMENT, 1201 AMGEN COURT WEST, SEATTLE, WA, 98119, US

(73) Assignee: Amgen Inc., Seattle, WA (US)

(21) Appl. No.: 12/072,737

(22) Filed: Feb. 27, 2008

Related U.S. Application Data
(63) Continuation of application No. 11/112,240, filed on Apr. 21, 2005, now abandoned.
(60) Provisional application No. 60/585,686, filed on Jul. 6, 2004.
(60) Provisional application No. 60/564,885, filed on Apr. 23, 2004.

(51) Int. Cl. A61K 39/395; C07K 16/28; C12N 15/13; C12N 15/85; C12N 5/16; C12P 21/02; C12N 5/00; A61P 35/00

(52) U.S. Cl. 424/133.1; 530/387.9; 530/387.3; 424/139.1; 536/23.53; 435/320.1; 435/328; 435/69.6; 435/377; 435/331

(57) ABSTRACT

The present invention provides compositions and methods relating to anti-CD148 receptor antibodies. Methods provided include inhibiting angiogenesis and, thereby, vascularization of solid tumors in human patients. The present invention also provides compositions and methods for in vivo imaging of tumors expressing CD148. Compositions of the invention include: anti-CD148 antibodies, antigen binding regions of anti-CD148 antibodies, polynucleotides encoding anti-CD 148 antibodies or binding regions thereof, vectors comprising these polynucleotides, host cells, and pharmaceutical compositions. Methods of making and using each of these compositions is also provided.

### CROSS-REFERENCE TO RELATED APPLICATIONS

This application is a continuation of U.S. application Ser. No. 11/112,240 filed Apr. 21, 2005 and claims the benefit under 35 U.S.C. §119(e) of U.S. Provisional Application Ser. No. 60/564,885 filed Apr. 23, 2004; and Ser. No. 60/585,686 filed Jul. 6, 2004, all of which are incorporated by reference herein.

### TECHNICAL FIELD OF THE INVENTION

The present invention relates to anti-CD148 antibodies for use in therapeutic and diagnostic applications.

### BACKGROUND OF THE INVENTION

CD148 is a mammalian transmembrane protein, also referred to as DEP-1 (density enhanced phosphatase), ECRTP (endothelial cell receptor tyrosine phosphatase), HPTPη, or BYP, depending upon species and cDNA origin. Human CD148 belongs to a class of endothelial cell surface receptors known as Type III density enhanced receptor protein tyrosine phosphatases (PTP). Protein tyrosine phosphorylation is an essential element in signal transduction pathways which control fundamental cellular processes including growth and differentiation, cell cycle progression, and cytoskeletal function. Binding of a ligand to a receptor protein tyrosine kinase (PTK) catalyzes autophosphorylation of tyrosine residues in the enzyme's target substrates, while binding of a ligand to a receptor PTP catalyzes dephosphorylation. The level of intracellular tyrosine phosphorylation of a target substrate is determined by the balance between PTK and PTP. PTKs play a significant role in promoting cell growth, while PTPs down-regulate the activity of PTKs by inhibiting cell growth. CD148 has been shown to promote differentiation of erythroid progentior cells, modulate lymphocyte function when crosslinked with other signaling proteins, and inhibit clonal expression of breast cancer cell lines overexpressing the protein. Confirming its role as an inhibitor of cell growth, CD 148 has also recently been shown to mediate inhibitory signals that block angiogenesis, an essential biological activity necessary for cell migration and proliferation, making CD 148 an important target for treatment of cancer by activating CD148 mediated inhibition of angiogenesis associated with tumor growth.

Like other receptor protein tyrosine phosphatases, CD148 has an intracellular carboxyl moiety with a catalytic domain, a single transmembrane domain, and an extracellular amino terminal domain (comprising at least five tandem fibronectin type III (FNIII) repeats, which have a folding pattern similar to that of Ig-like domains). The FNIII domains have an absolute specificity for phosphotyrosine residues, a high affinity for substrate proteins, and a specific activity which is several orders of magnitude greater than that of the PTKs. The FNIII domains are believed to participate in protein/protein interactions. Activation of CD148 triggers autophosphorylation of CD148, which tranduces a biological signal resulting in inhibition of angiogenesis.

U.S. Pat. No. 6,552,169 discloses polynucleotide sequences relating to human DEP-1 (CD148) and polyclonal antibodies generated against polypeptides encoded by the polynucleotides. U.S. Pat. No. 6,248,327 discloses the role of CD148 in angiogenesis and provides a method of modulating angiogenesis in a mammal by administering compositions that specifically bind to the ectodomain of CD148, and also discloses the use of monoclonal antibodies that specifically bind to an unspecified region of the CD148 ectodomain to activate CD148 anti-angiogenesis activity.

In view of the role of angiogenesis in the growth of solid tumors and other diseases, the development of improved therapeutic agents that activate CD148 anti-angiogenesis activity would represent a significant advance in cancer therapeutic modalities.

### SUMMARY OF THE INVENTION

The present invention provides an isolated antibody or an antigen binding region thereof, having a polypeptide sequence having at least 90% sequence identity to a variable chain sequence of an exemplary reference antibody (one of Ab-1 through Ab-8) of the present invention. The antibody or antigen binding region specifically binds to the extracellular domain of human CD148. In some embodiments the isolated antibody or an antigen binding region is competitively inhibited from specifically binding to human CD148, to a statistically significant degree, by antibodies having a variable heavy chain and a variable light chains of one of the reference antibodies of the present invention. In some embodiments the isolated antibody or an antigen binding region has a heavy chain variable polypeptide sequence and a light chain variable polypeptide sequence with at least 90% sequence identity to the heavy and light chains of one of Ab-1 through Ab-8. In other embodiments the sequence identity is 100%. In some embodiments, specific binding yields at least 10% inhibition as measured an HRMEC human renal microvascular endothelial cell planar migration assay. An antibody of the invention can be a human IgG₂ isotype. In some embodiments, the antigen binding region of the invention is an Fab, F(ab′)₂, Fv, scFv, or dimerized scFV fused to the Fc of IgG₁.

In some aspects of the invention, the antibody or isolated antigen binding regions are competitively inhibited from specifically binding, to a statistically significant degree, by antibodies having the variable chains of at least one of Ab-1 through Ab-8. The isolated antibody or antigen binding region can be covalently bonded to a conjugate. Often, the antibodies or antigen binding regions which is human or humanized in which case they can also be in a carrier pharmaceutically acceptable for administration in humans. Often, the antibody or antigen binding region or combination thereof is admixed with a carrier pharmaceutically acceptable in humans at a concentration of at least around 1 microgram per milliliter. In some embodiments present invention is directed to kits including an antibody or antigen binding region of the present invention in a carrier that is pharmaceutically acceptable in humans. This pharmaceutical composition is provided sealed within a sterile container and including a package insert with written instructions on dosage of the pharmaceutical composition.

In some aspects of the invention, the isolated antibody or antigen binding region has at least one complementarity determining regions (CDRs) from the variable chains of Ab-1 through Ab-8. These antibodies or antigen binding regions will specifically bind to the extracellular domain of human CD148. In some embodiments, the antibodies or antigen binding regions will have both the VH polypeptide sequence and its cognate VL polypeptide sequence for one of the three CDR pairs (i.e., CDR1, CDR2, or CDR3) present in one of Ab-1 through Ab-8. Often, two such CDR pairs will be present.

Nucleic acids having polynucleotides which encode each of the aforementioned embodiments are also provided, as well as expression vectors and host cells (e.g., CHO cells) for same. Methods of transfecting, expressing the vectors of the invention, and isolating the antibody or antigen binding region compositions of the invention are additional aspects of the invention. The antibody or antigen binding region of the invention can be covalently linked, directly or indirectly, to a conjugate which can be a detectable label, a cytotoxic agent, a lipid, polyethylene glycol, or a carbohydrate.

In a further aspect, the invention provides a method of inhibiting angiogenesis, in a human subject, of angiogenically active vascular endothelial cells expressing a CD148 receptor, which entails administering to a human a therapeutically effective amount of an antibody or antigenic binding region pharmaceutical composition and inhibiting angiogenesis. In certain therapeutic regimens, the angiogenically active vascular endothelial cells to be inhibited provide a blood supply to a solid tumor or to inflamed tissue.

### BRIEF DESCRIPTION OF THE DRAWINGS

FIGS. 1A and 1B show a nucleotide and encoded amino acid sequence overlap for variable heavy (VH) and variable light (VL) chains for an antibody of the present invention, Antibody No. 1 (Ab-1). Shaded regions on the figure highlight CDR1, 2, and 3 (from amino to carboxy terminus, respectively). Query and Frame1 designations indicate the nucleotide and amino acid sequences, respectively.

FIGS. 2A and 2B show a nucleotide and encoded amino acid sequence overlap for variable heavy (VH) and variable light (VL) chains for Antibody No. 2 (Ab-2). Shaded regions on the figure highlight CDR1, 2, and 3 (from amino to carboxy terminus, respectively).

FIGS. 3A and 3B show a nucleotide and encoded amino acid sequence overlap for variable heavy (VH) and variable light (VL) chains for Antibody No. 3 (Ab-3). Shaded regions on the figure highlight CDR1, 2, and 3 (from amino to carboxy terminus, respectively).

FIGS. 4A and 4B show a nucleotide and encoded amino acid sequence overlap for variable heavy (VH) and variable light (VL) chains for Antibody No. 4 (Ab-4). Shaded regions on the figure highlight CDR1, 2, and 3 (from amino to carboxy terminus, respectively).

FIGS. 5A and 5B show a nucleotide and encoded amino acid sequence overlap for variable heavy (VH) and variable light (VL) chains for Antibody No. 5 (Ab-5). Shaded regions on the figure highlight CDR1, 2, and 3 (from amino to carboxy terminus, respectively).

FIGS. 6A and 6B show a nucleotide and encoded amino acid sequence overlap for variable heavy (VH) and variable light (VL) chains for Antibody No. 6 (Ab-6). Shaded regions on the figure highlight CDR1, 2, and 3 (from amino to carboxy terminus, respectively).

FIGS. 7A and 7B show a nucleotide and encoded amino acid sequence overlap for variable heavy (VH) and variable light (VL) chains for Antibody No. 7 (Ab-7). Shaded regions on the figure highlight CDR1, 2, and 3 (from amino to carboxy terminus, respectively).

FIGS. 8A and 8B show a nucleotide and encoded amino acid sequence overlap for variable heavy (VH) and variable light (VL) chains for Antibody No. 8 (Ab-8). Shaded regions on the figure highlight CDR1, 2, and 3 (from amino to carboxy terminus, respectively).

### DETAILED DESCRIPTION OF THE INVENTION

The present invention provides compositions and methods relating to anti-CD148 receptor antibodies, including methods for treating in human subjects certain conditions involving CD148, such methods include inhibiting angiogenesis and, thereby, vascularization of solid tumors. The present invention also provides compositions and methods for in vivo imaging of tumors expressing CD148. Compositions of the invention include: anti-CD148 antibodies, antigen binding regions of CD148 antibodies, polynucleotides encoding anti-CD148 antibodies or binding regions thereof, vectors comprising these polynucleotides, host cells comprising and host cells expressing these vectors, and pharmaceutical compositions. Methods of making and using each of these compositions is also provided.

### A. Definitions

Units, prefixes, and symbols may be denoted in their SI accepted form. Unless otherwise indicated, nucleic acids are written left to right in 5′ to 3′ orientation; amino acid sequences are written left to right in amino to carboxy orientation. Numeric ranges recited herein are inclusive of the numbers defining the range and include and are supportive of each integer within the defined range. Amino acids may be referred to herein by either their commonly known three letter symbols or by the one-letter symbols recommended by the IUPAC-IUBMB Nomenclature Commission. Nucleotides, likewise, may be referred to by their commonly accepted single-letter codes. Unless otherwise noted, the terms “a” or “an” are to be construed as meaning “at least one of”. The section headings used herein are for organizational purposes only and are not to be construed as limiting the subject matter described. All documents, or portions of documents, cited in this application, including but not limited to patents, patent applications, articles, books, and treatises, are hereby expressly incorporated by reference. In the case of any amino acid or nucleic sequence discrepancy within the application, the figures control.

As used herein, the term “antibody” includes reference to both glycosylated and non-glycosylated immunoglobulins of any isotype or subclass, including human (e.g., CDR-grafted), humanized, chimeric, multi-specific, monoclonal, polyclonal, and oligomers thereof, irrespective of whether such antibodies are produced, in whole or in part, via immunization, through recombinant technology, by way of in vitro synthetic means, or otherwise. Thus, the term “antibody” in inclusive of those that are prepared, expressed, created or isolated by recombinant means, such as (a) antibodies isolated from an animal (e.g., a mouse) that is transgenic for human immunoglobulin genes or a hybridoma prepared therefrom, (b) antibodies isolated from a host cell transfected to express the antibody (e.g., from a transfectoma), (c) antibodies isolated from a recombinant, combinatorial antibody library, and (d) antibodies prepared, expressed, created or isolated by any other means that involve splicing of immunoglobulin gene sequences to other DNA sequences. Such antibodies have variable and constant regions derived from germline immunoglobulin sequences of two distinct species of animals. In certain embodiments, however, such antibodies can be subjected to in vitro mutagenesis (or, when an animal transgenic for human immunoglobulin sequences is used, in vivo somatic mutagenesis) and thus the amino acid sequences of the VH and VL regions of the antibodies are sequences that, while derived from and related to the germline VH and VL sequences of a particular species (e.g., human), may not naturally exist within that species' antibody germline repertoire in vivo.

As used herein, the term “antigen binding region” refers to a fragment of an antibody or a polypeptide which has at least 1 (e.g., 1, 2, 3, or more) heavy chain sequences and/or at least 1 (e.g., 1, 2, 3, or more) light chain sequences for a particular complementarity determining region (CDR) (i.e., at least one of CDR1, CDR2, and/or CDR3 from the heavy and/or light chain). Exemplary antigen binding regions include: F(ab), F(ab′)₂, Fv, diabodies, Fd (consisting of the VH and CH1 domains), maxibodies (bivalent scFV fused to the amino terminus of the Fc (CH2—CH3 domains) of IgG₁), and single chain antibody molecules, including single-chain FV (scFv), see e.g., Bird et al. (1988) Science 242:423-426; and Huston et al. (1988) Proc. Natl. Acad. Sci. USA 85:5879-5883). Fusions of CDR containing polypeptide sequences to an Fc region (or a constant heavy 2 (CH2) or constant heavy 3 (CH3) containing region thereof) are included within the scope of this definition including, for example, scFV fused, directly or indirectly (e.g. through a chemical spacer), to an Fc are included herein. An antigen binding region is inclusive of, but not limited to, those derived from an antibody or fragment thereof (e.g., by enzymatic digestion or reduction of disulfide bonds), produced synthetically using recombinant methods (e.g., transfectomas), created via in vitro synthetic means (e.g., polypeptide synthesis using Merrifield resins), combinations thereof, or through other methods. Thus, antigen binding regions of the present invention include polypeptides produced by any number of methods which comprise at least one CDR from a VH or VL chain of the present invention (e.g., Ab-1 through Ab-8).

The term “CDR-grafted” refers to an antibody or antigen binding region in which the CDRs derived from one species are inserted into the framework of a different species, such as murine CDRs grafted on a human framework (a “human” antibody).

The term “chimeric antibody” refers to an antibody in which a portion of the antibody is homologous to a sequence of a particular species or a particular antibody class, while another portion of the antibody is homologous to a sequence of a different species or antibody class. See, e.g., U.S. Pat. No. 4,816,567 and Morrison et al., Proc Natl Acad Sci (USA), 81:6851-6855 (1985).

By “competitively inhibit” is meant that an antibody or antigen binding region inhibits, to a statistically significant degree, the specific binding to the same, or substantially the same, epitope as another antibody or antigen binding portion thereof. Typically, competitive inhibition is measured by determining the amount of a reference antibody or antigen binding region which is bound to the target protein (e.g., human CD148) in the presence of the tested antibody or antigen binding region thereof. Usually the tested antibody or tested antigen binding region is present in excess, such as 5-, 10-, 25-, or 50-fold excess. Competitively bound antibodies or antigen binding regions will, when present in excess, inhibit specific binding of a reference antibody or antigen binding region to the extracellular domain of human CD148 by a statistically significant degree, often at least 10%, 25%, 50%, 75%, 90% or greater. Competitive inhibition assays are well known in the art. See, for example, Harlow and Lane (1998), Antibodies, A Laboratory Manual, Cold Spring Harbor Publications, New York.

As used herein, “conjugate” means any chemical or biological moiety that, when conjugated to an antibody or antigen binding region, serves as a detectable label, or acts to substantially increase the pharmacokinetic or pharmacodynamic properties of the antibody or antigen binding region to which it is directly or indirectly (i.e., through a chemical spacer) covalently attached. Exemplary conjugates include: cytotoxic or cytostatic agents, polyethylene glycol, anti-angiogenic agents, and lipids.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational (or linear) epitopes are distinguished in that the binding to the former but not the latter is lost in the presence of denaturing solvents.

A “host cell” is a cell that can be used to express a nucleic acid, e.g., a nucleic acid of the present invention. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Examples of host cells include the COS-7 line of monkey kidney cells (ATCC CRL 1651) (see Gluzman et al., 1981, Cell 23:175), L cells, C 127 cells, 3T3 cells (ATCC CCL 163), Chinese hamster ovary (CHO) cells or their derivatives such as Veggie CHO and related cell lines which grow in serum-free media (see Rasmussen et al., 1998, Cytotechnology 28:31) or CHO strain DX-B11, which is deficient in DHFR (see Urlaub et al., 1980, Proc. Natl. Acad. Sci. USA 77:4216-20), HeLa cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Typically, a host cell is a cultured cell that can be transfected with a polypeptide-encoding nucleic acid, which can then be expressed in the host cell. The phrase “recombinant host cell” can be used to denote a host cell that has been transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “human antibody” refers to an antibody in which both the constant regions and the framework consist of fully or substantially human sequences such that the human antibody elicits substantially no immunogenic reaction against itself when administered to a human host and preferably, no detectable immunogenic reaction. In certain embodiments, human antibodies are produced in non-human mammals, including, but not limited to, mice, rats, and lagomorphs. In certain embodiments, human antibodies are produced in hybridoma cells from transgenic animals having a human immunoglobulin repertoire. In certain embodiments, fully human antibodies are produced recombinantly, such as in a transfectoma.

The term “humanized antibody” refers to an antibody in which substantially all of the constant region is derived from a human, while all or part of one or more variable regions is derived from another species, for example a mouse.

As used herein, “human CD 148” is the protein identified as human ECRTP/DEP-1 in Ostman et al., Proc Natl Acad Sci USA 91:9680-9684 (1994), incorporated by reference herein, including allelic variants thereof. By “extracellular domain of human CD148” is meant the portion of human CD148 localized between about residues 36 to 973 (residues 1 to 35 being the leader sequence and not present in the mature form) of NCBI (National Center for Biotechnology Information) accession AAB36687 version AAB36687.1 GI:1685075, submitted Nov. 26, 1996, incorporated by reference herein and available on the world wide web at ncbi.nlm.nih.gov.

As used herein, “inhibits angiogenesis” means a statistically significant reduction in the level of angiogenesis relative to an untreated control. Exemplary reductions are from at least 5 to 99%, and thus include at least 10%, 20%, 30%, 40%, 50%, 60%, 70%, 80%, or 90% reduction in angiogenesis relative to a negative control. Widely accepted functional assays of angiogenesis such as the corneal micropocket assay and the human renal microvascular endothelial cell (HRMEC) planar migration assay are known in the art. See, e.g., U.S. Pat. Nos. 5,712,291 and 5,871,723. Briefly, an HRMEC planar migration assay is a wound closure that assay can be used to quantitate the inhibition of angiogenesis by antibodies or antigen binding regions of the present invention in vitro. In this assay, endothelial cell migration is measured as the rate of closure of a circular wound in a cultured cell monolayer. The rate of wound closure is linear, and is dynamically regulated by agents that stimulate and inhibit angiogenesis in vivo.

As those of ordinary skill in the art are aware, a mouse corneal pocket assay can also be used to quantitate the inhibition of angiogenesis by antibodies or antigen binding regions of the present invention in vivo. In this assay, agents to be tested for angiogenic or anti-angiogenic activity are immobilized in a slow release form in a hydron pellet, which is implanted into micropockets created in the corneal epithelium of anesthetized mice. Vascularization is measured as the appearance, density, and extent of vessel ingrowth from the vascularized corneal limbus into the normally avascular cornea. See, U.S. Pat. No. 6,248,327 which describes planar migration and corneal pocket assays.

As used herein, “isolated” in the context of a nucleic acid means DNA or RNA which as a result of direct human intervention: 1) is integrated into a locus of a genome where it is not found in nature, 2) is operably linked to a nucleic acid to which it is not operably linked to in nature, or, 3) is substantially purified (e.g., at least 70%, 80%, or 90%) away from cellular components with which it is admixed in its native state.

The term “isolated” in the context of an antibody means: (1) is substantially purified (e.g., at least 60%, 70%, 80%, or 90%) away from cellular components with which it is admixed in its endogenously expressed native state such that it is the predominant species present, (2) is conjugated to a polypeptide or other entity to which it is not linked in nature, (3) does not occur in nature as part of a larger polypeptide sequence, (4) is combined with other antibodies or agents having different specificities in a well-defined composition, or (5) comprises a human engineered sequence not otherwise found in nature.

The terms “monoclonal antibody” or “monoclonal antibody composition” as used herein refer to a preparation of antibody molecules of single molecular composition, typically encoded by the same nucleic acid molecule. A monoclonal antibody composition displays a single binding specificity and affinity for a particular epitope. In certain embodiments, monoclonal antibodies are produced by a single hybridoma or other cell line (e.g., a transfectoma), or by a transgenic mammal. Monoclonal antibodies typically recognize the same epitope. The term “monoclonal” is not limited to any particular method for making an antibody.

The term “multi-specific antibody” refers to an antibody wherein two or more variable regions bind to different epitopes. The epitopes may be on the same or different targets. In certain embodiments, a multi-specific antibody is a “bispecific antibody,” which recognizes two different epitopes on the same or different antigens.

As used herein, “nucleic acid” includes reference to a deoxyribonucleotide or ribonucleotide polymer, or chimeras thereof, and unless otherwise limited, encompasses the complementary strand of the referenced sequence.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid. Thus, a regulatory sequence and a second sequence are operably linked if a functional linkage between the regulatory sequence and the second sequence is such that the regulatory sequence initiates and mediates transcription of the DNA sequence corresponding to the second sequence. Examples of regulatory sequences include promoters, enhancers and other expression control elements (e.g., polyadenylation signals). Further examples of regulatory sequences are described in, for example, Goeddel, 1990, Gene Expression Technology Methods in Enzymology 185, Academic Press, San Diego, Calif. and Baron et al., 1995, Nucleic Acids Res. 23:3605-06.

The terms “peptide,” “polypeptide” and “protein” are used interchangeably throughout and refer to a molecule comprising two or more amino acid residues joined to each other by peptide bonds. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation.

The term “polyclonal antibodies” refers to a heterogeneous mixture of antibodies that bind to different epitopes of the same antigen.

The terms “polynucleotide,” “oligonucleotide” and “nucleic acid” are used interchangeably throughout and include DNA molecules (e.g., cDNA or genomic DNA), RNA molecules (e.g., mRNA), and hybrids thereof. The nucleic acid molecule can be single-stranded or double-stranded.

As used herein, “sequence identity” is the value obtained by comparing two polynucleotide or polypeptide sequences is determined by using the GAP computer program (a part of the GCG Wisconsin Package, version 10.3 (Accelrys, San Diego, Calif.)) using its default parameters.

As used herein, “specifically binds” or “specifically binding” or “binds specifically” refers to binding reaction which is determinative of the presence of the target (e.g., a protein) in the presence of a heterogeneous population of proteins and other biologics. Thus, under designated immunoassay conditions, the specified antibodies or binding regions thereof, bind to a particular protein and do not bind in a statistically significant amount to other proteins present in the sample. Typically, antibodies or binding regions thereof, are selected for their ability to specifically bind to a protein by screening methods (e.g., phage display) or by immunization using the protein or an epitope thereof. See, Harlow and Lane (1998), Antibodies, A Laboratory Manual, Cold Spring Harbor Publications, New York, for a description of immunoassay formats that can be used to determine specific binding. For example, solid-phase ELISA immunoassays can be used to determine specific binding. Specific binding proceeds with an association constant of at least about 1×10⁷ M⁻¹, and often at least 1×10⁸ M⁻¹, 1×10⁹ M⁻¹, or, 1×10¹⁰ M⁻¹.

The term “stringent conditions” or “stringent hybridization conditions” means conditions under which a nucleic acid will hybridize to its target sequence, to a statistically significant and detectably greater degree than to other sequences (e.g., at least 2-fold over background). Sequences that bind to a target under stringent conditions are selective for the target (“selectively hybridize”). Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of identity are detected.

The term “variable” in the context of variable light and heavy chains of an antibody, refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

As used herein, “vector” includes reference to a nucleic acid used in the introduction of a polynucleotide of the present invention into a host cell. Vectors are often replicons. Expression vectors permit transcription of a nucleic acid inserted therein.

### B. Antibodies and Antigen Binding Regions

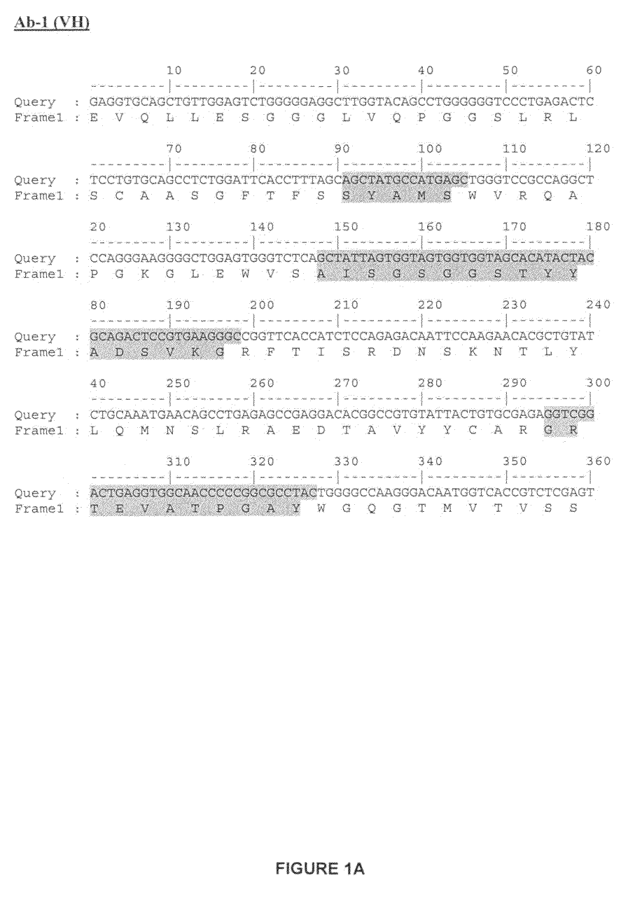

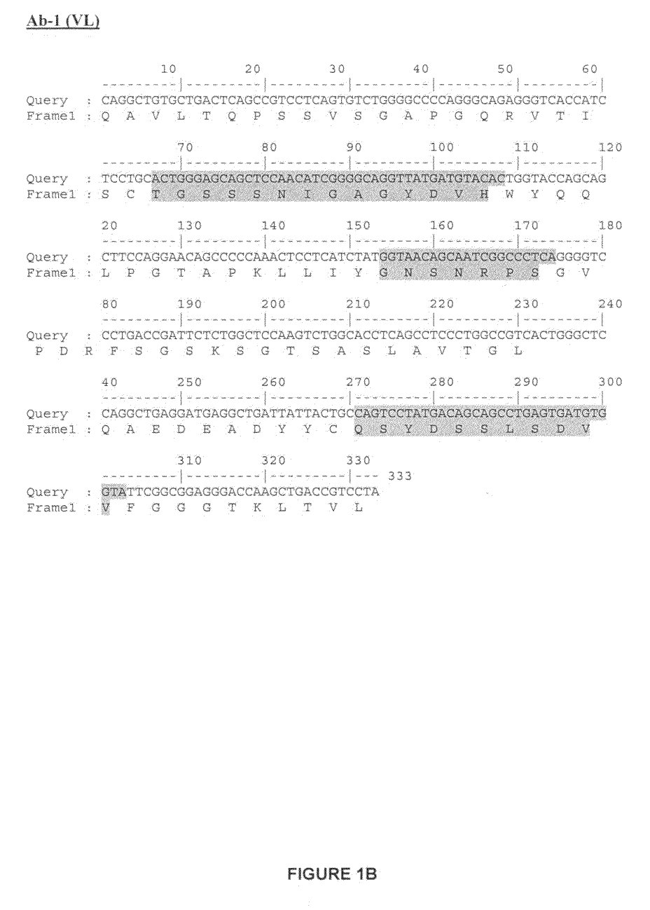

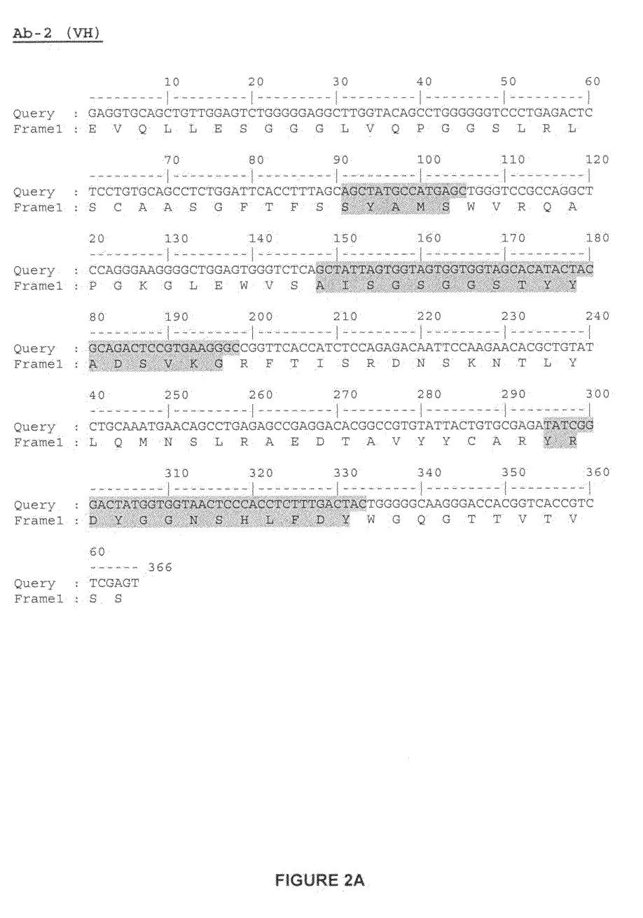

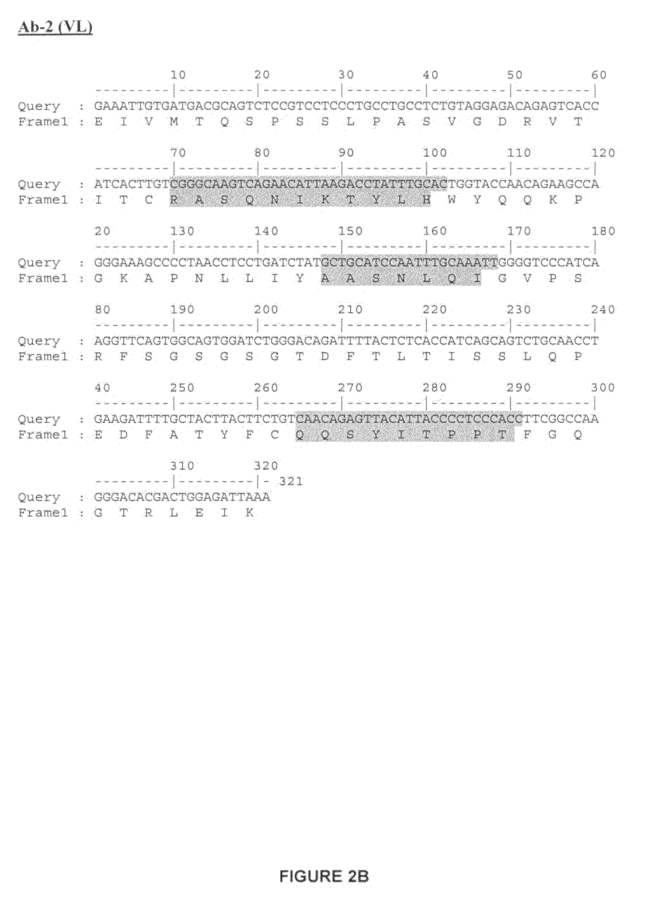

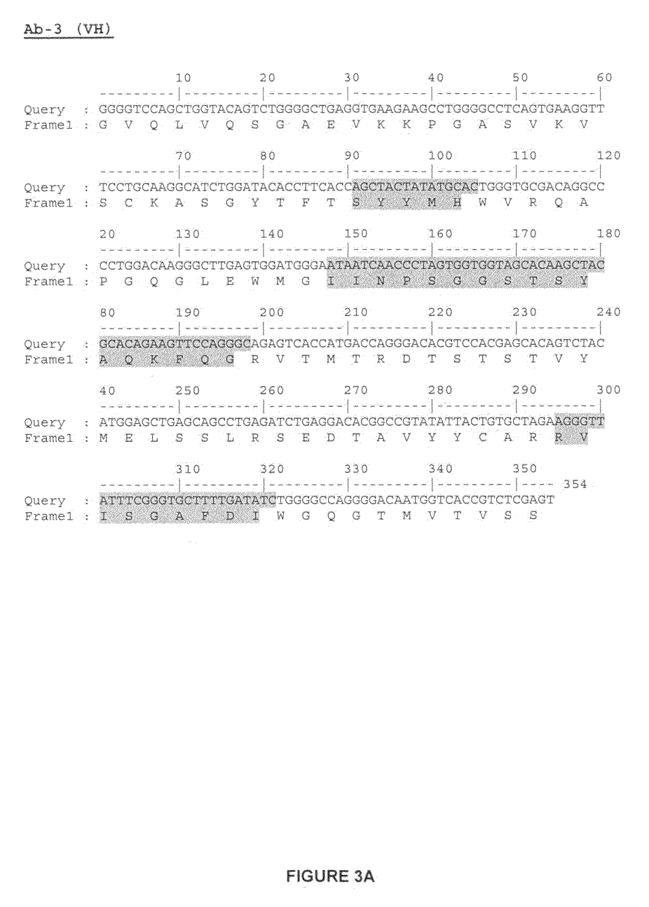

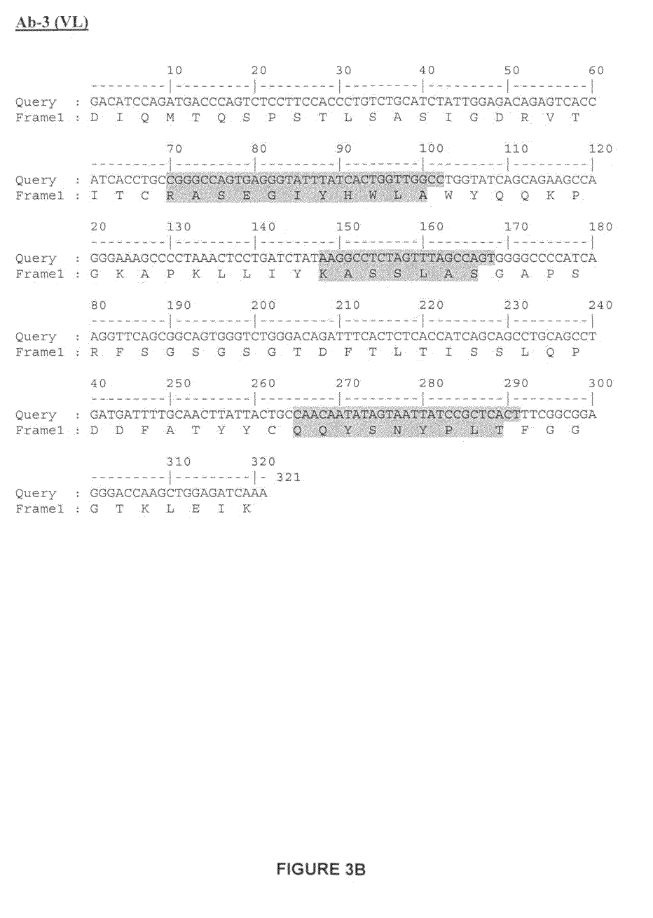

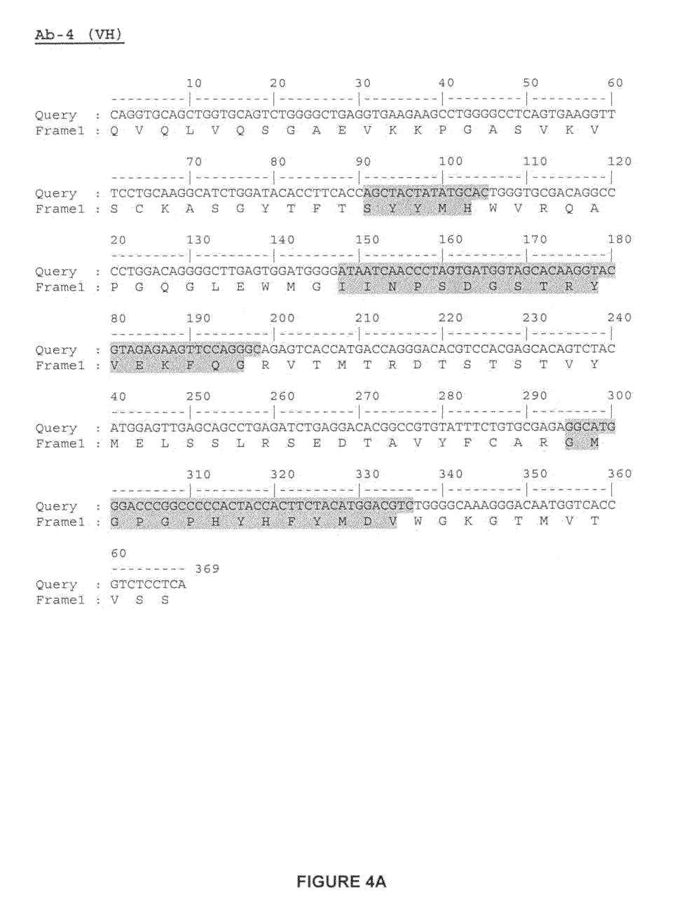

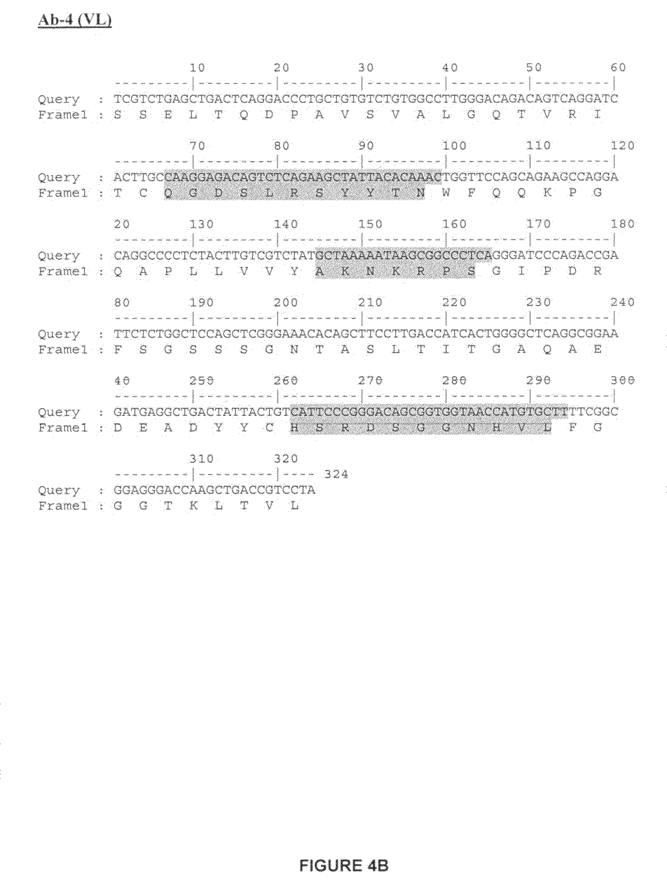

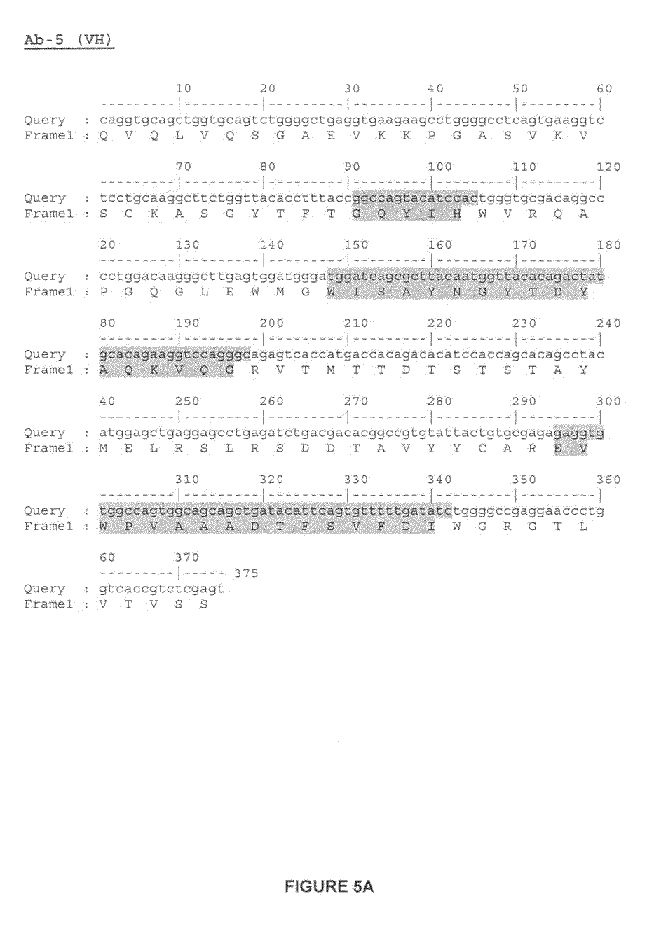

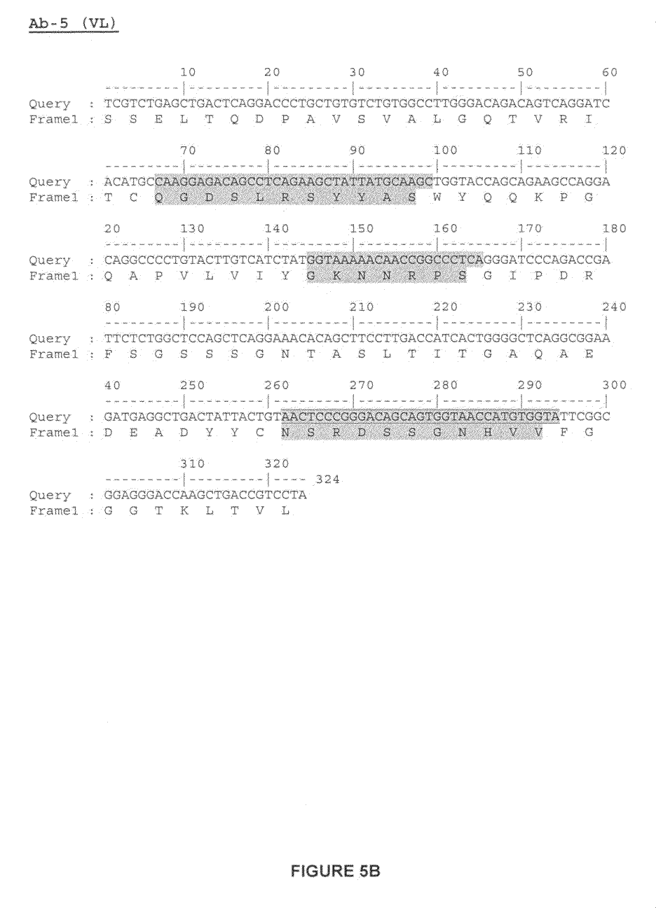

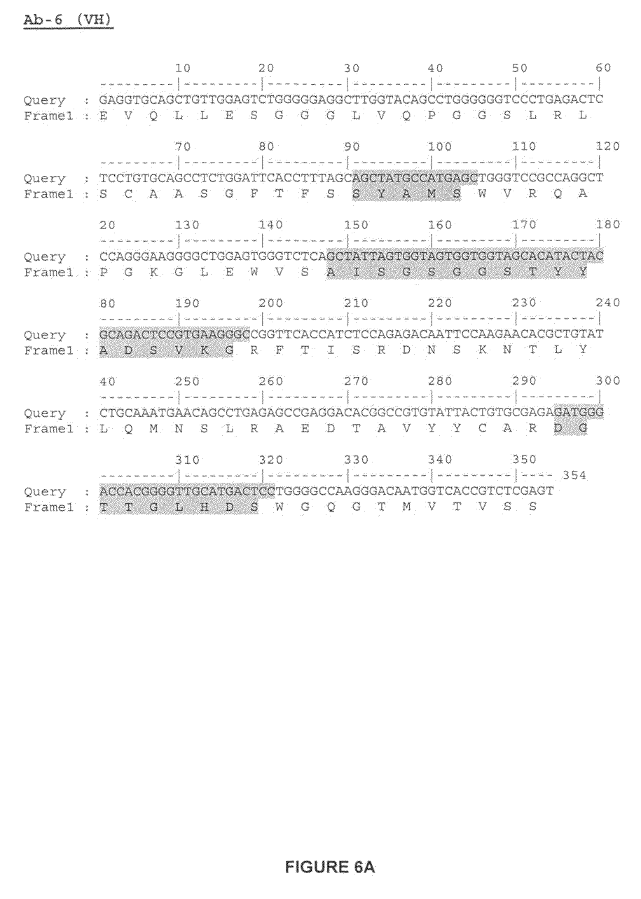

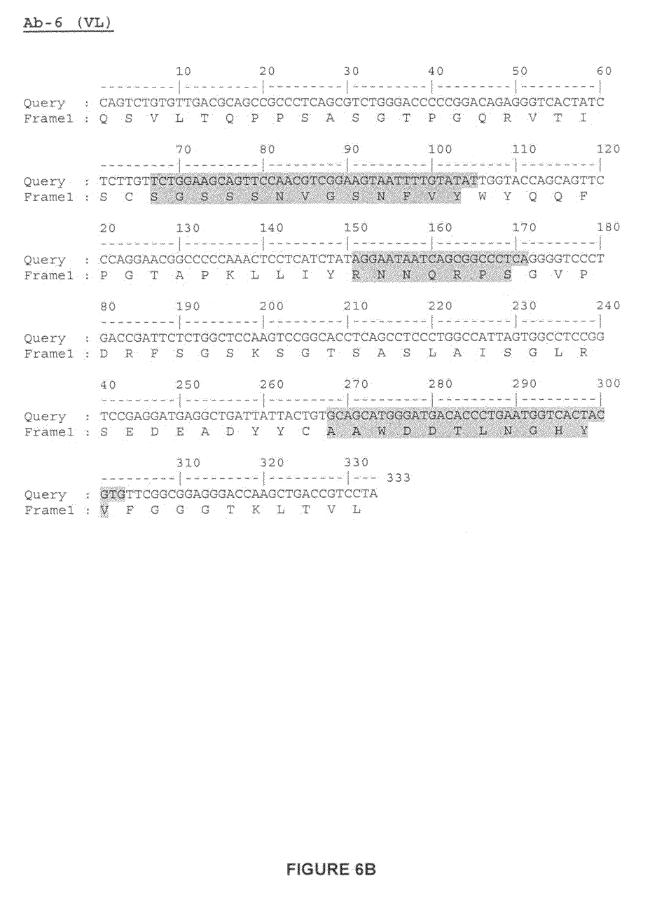

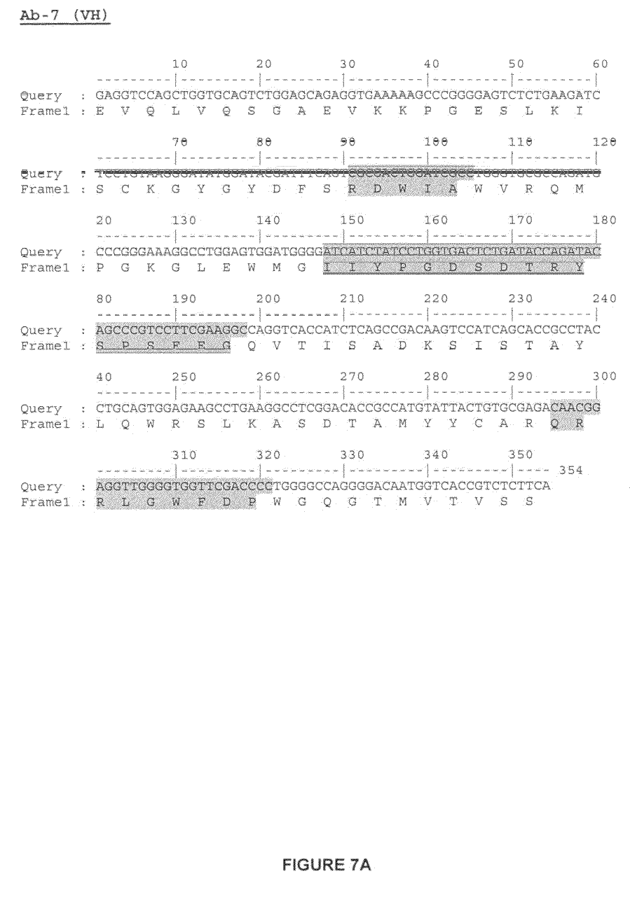

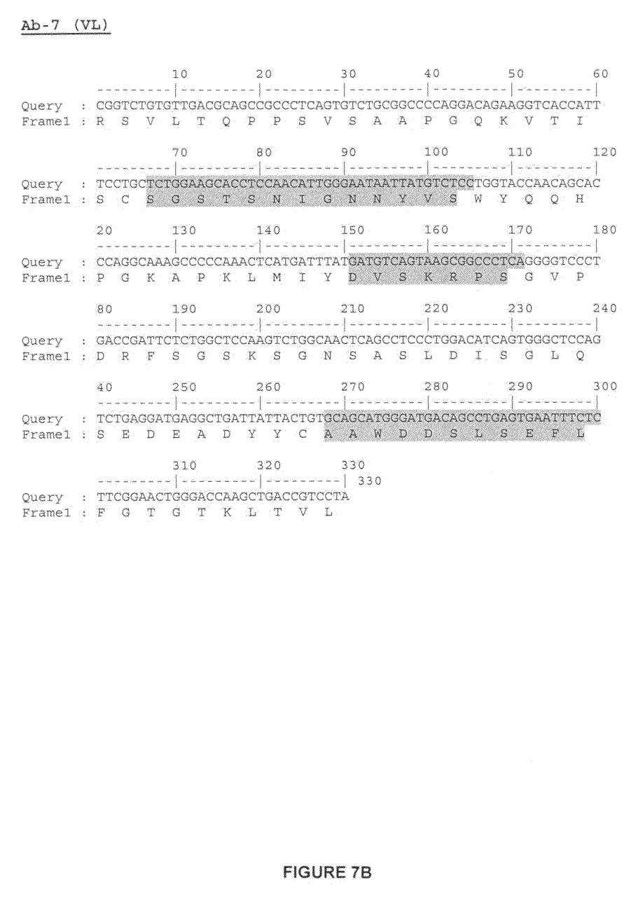

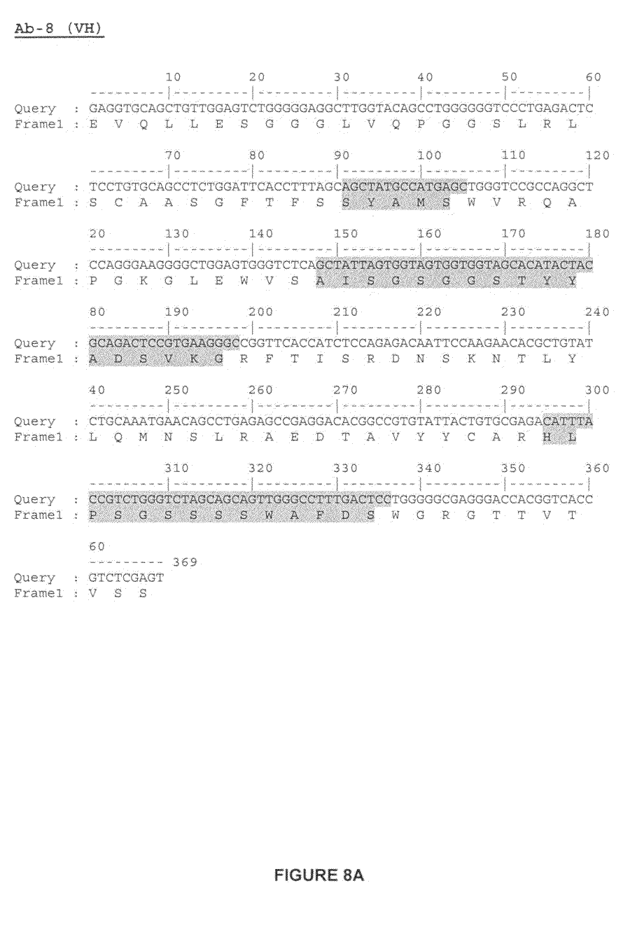

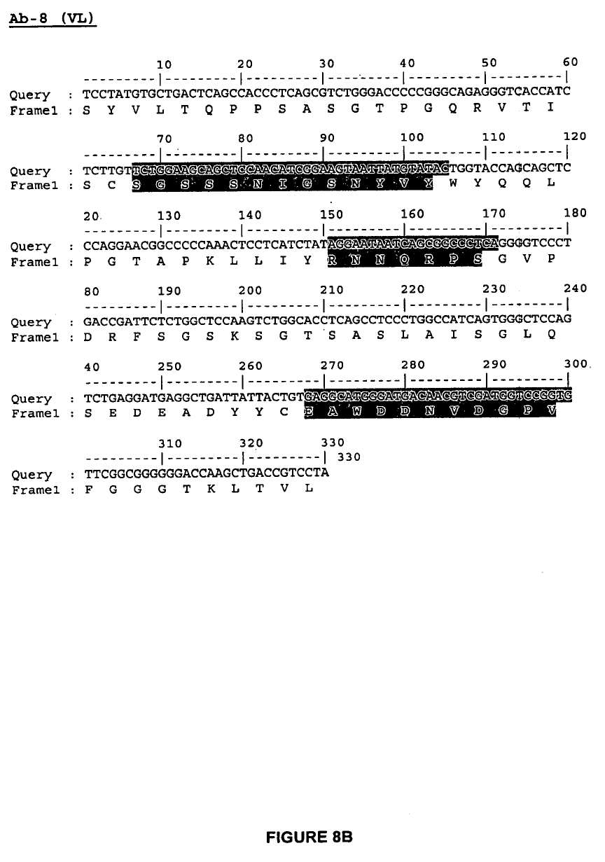

The present invention provides variable heavy and variable light chain polypeptide sequences (Ab-1, Ab-2, Ab-3, Ab-4, Ab-5, Ab-6, Ab-7, and Ab-8, collectively, “Ab-1 through Ab-8”) for isolated antibodies and antigen binding regions of the present invention. Those of skill in the art will recognize that the variable heavy and variable light pairs of each of Ab-1 through Ab-8 can be used within different heavy chain isotypes—IgG, IgM, IgD, IgA, and IgE, and within different subclasses of each isotype, each of which is encompassed by the present invention. Frequently, however, an antibody of the present invention will be an IgG antibody, such as a human IgG₂ antibody or a maxibody (bivalent scFVs covalently attached to the Fc region of IgG₁, see, Fredericks et al, Protein Engineering, Design & Selection, 17:95-106 (2004); Powers et al., Journal of Immunological Methods, 251:123-135 (2001), see Shu et al., “Secretion of single-gene-encoded immunoglobulin from myeloma cells,” PNAS 90:7995-7999 (1993). Hayden et al., “Single-chain mono- and bispecific antibody derivatives with novel biological properties and antitumor activity from a COS cell transient expression system,” Therapeutic Immunology 1:3-15 (1994)). The heavy and variable light chains for each desired antibody structure (Ab-1 through Ab-8) are provided in FIGS. 1-8, respectively.

Antigen binding regions of each of Ab-1 through Ab-8 are also included within the scope of the present invention. Antigen binding regions are inclusive of those comprising at least one and, in some embodiments, 2, 3, 4, 5, or 6 distinct CDRs from one of Ab-1 through Ab-8. Any such combination of CDRs from the VH and/or VL chains of Ab-1 through Ab-8 are embraced within the scope of the present invention. Often, the CDRs will be from a single heavy and light variable chain pair of any one of Ab-1 through Ab-8. In some embodiments, the antigen binding region of the present invention will comprise a “CDR pair”, one CDR from the VH (variable heavy) chain and one CDR from the VL (variable light) chain from the same member of the group consisting of Ab-1 through Ab-8, where each CDR of the pair is of a specified type (CDR1, CDR2, or CDR3). Thus, for example, antigen binding regions will often comprise both CDR3 (VH) and CDR3 (VL) from at least one of: Ab-1, Ab-2, Ab-3, Ab-4, Ab-5, Ab-6, Ab-7, or Ab-8. Often antigen binding regions will comprise two distinct CDR pairs, each pair is typically, but not necessarily, from the same heavy and light chain pairs provided in Ab-1 through Ab-8. In some embodiments, antigen binding regions will comprise three distinct CDR pairs (i.e., CDR1 (VH & VL), CDR2 (VH & VL), and CDR3 (VH & VL), each of which is from the same or from a distinct member of the group of Ab-1 through Ab-8. Antigen binding regions comprising multiple identical CDRs are also included herein. The appropriate number and combination of VH and VL CDR sequences can be determined by those skilled in the art depending on the desired affinity and specificity and the intended use of the antibody or antigen binding region comprising the CDRs.

Sequence identity variations in the amino acid sequences of antibodies and antigen binding regions are encompassed by the present invention, providing that the variations in the amino acid sequence maintain at least from 75% to 99% sequence identity to an antibody or antigen binding region of the present invention as determined by the GAP program (a part of the GCG Wisconsin Package, version 10.3 (Accelrys, San Diego, Calif.) under default parameters. Exemplary sequence identity values are at least 80%, 85%, 90%, 93%, 95%, 97%, or 99% sequence identity. In some embodiments, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar-glycine, asparagine, glutamine, cysteine, serine, threonine, tyrosine. Often, families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. In some embodiments, the number of conservative substitutions per 100 residues is from 1 to 15, often 1, 2, 3, 4, or 5 conservative substitutions. Conservative substitutions can be made throughout the variable heavy and/or light chains or limited to the CDRs thereof.

In some embodiments, the antibodies and antigen binding regions of the present invention and sequence identity variants of these, as discussed above, will be limited to those able to specifically bind to human CD148, typically the extracellular domain of human CD148 in its native conformation. In some embodiments, antibodies or antigen binding regions of the present invention will be competitively inhibited from specifically binding by at least one antibody or antigen binding region (i.e., the test antibody and the test antigen binding region, respectively) thereof having variable heavy and variable light chains selected from one of Ab-1 through Ab-8 (the reference antibodies). Often the test antibody will be IgG isotype such as IgG₂. Thus, for example, competitive immunoassays can be employed to determine whether antibodies or antigen binding regions bind to substantially the same epitope as antibodies with VH and VL chains of one of Ab-1 through Ab-8.

### C. Nucleic Acids

(1) The present invention provides, among other things, isolated nucleic acids comprising a polynucleotide of the present invention. A polynucleotide of the present invention is inclusive of those encoding each antibody and antigen binding region, as well as sequence variants thereof, as disclosed in B, above, without limit. Antibodies and antigen binding regions comprising variable heavy and variable light chains are generically exemplified in Ab-1 through Ab-8 (FIGS. 1-8). Those of skill will recognize that the variable heavy and variable lights therein disclosed can be used in the engineering or synthesis of a wide variety of antibody isotypes and subclasses. Variable heavy and variable light chain polynucleotides are, respectively: for Ab-1 (SEQ ID NOS: 1 and 3) Ab-2 (SEQ ID NOS: 5 and 7), Ab-3 (SEQ ID NOS: 9 and 11), Ab-4 (SEQ ID NOS: 13 and 15), Ab-5 (SEQ ID NOS: 17 and 19), Ab-6 (SEQ ID NOS: 21 and 23), Ab-7 (SEQ ID NOS: 25 and 27), and, Ab-8 (SEQ ID NOS: 29 and 31).

(2) Isolated nucleic acids of the present invention also include those comprising a polynucleotide encoding an antibody or antigen binding region thereof, as disclosed in B, above. As will be understood by those of ordinary skill in the art, nucleic acid sequences herein that encode a polypeptide also, by reference to the genetic code, describes every possible silent variation of the nucleic acid. Each codon in a nucleic acid (except AUG, which is ordinarily the only codon for methionine; and UGG, which is ordinarily the only codon for tryptophan) can be modified to yield a functionally identical molecule. Thus, each silent variation of a nucleic acid which encodes a polypeptide of the present invention is implicit in each described antibody or antigen binding region sequence and is within the scope of the present invention. Accordingly, the present invention includes isolated nucleic acids encoding the heavy and light polypeptide chains, respectively: for Ab-1 (SEQ ID NOS: 2 and 4,) Ab-2 (SEQ ID NOS: 6 and 8,), Ab-3 (SEQ ID NOS: 10 and 12), Ab-4 (SEQ ID NOS: 14 and 16), Ab-5 (SEQ ID NOS: 18 and 20), Ab-6 (SEQ ID NOS: 22 and 24), and Ab-7 (SEQ ID NOS: 26 and 28), and for each heavy and light chain CDR thereof.

The polypeptides and polynucleotides of the present invention can be modified to alter codon usage. Altered codon usage can be employed to alter translational efficiency and/or to optimize the coding sequence for expression in a desired host such as to optimize the codon usage in specific host cells, such as CHO cells. Codon usage in the coding regions of the polynucleotides of the present invention can be analyzed statistically using commercially available software packages such as “Codon Preference” available from the University of Wisconsin Genetics Computer Group (see Devereaux et al., Nucleic Acids Res. 12: 387-395 (1984)) or MacVector 4.1 (Eastman Kodak Co., New Haven, Conn.).

(3) The present invention further provides isolated nucleic acids comprising polynucleotides of the present invention, wherein the polynucleotides hybridize, under stringent hybridization conditions, to a polynucleotide of sections C. (1) or C. (2), as discussed above. In some embodiments, the polynucleotides which hybridize under stringent conditions also encode antibodies or antigen binding regions which also specifically bind to the extracellular domain of human CD148. In other embodiments, the polynucleotides that hybridize under stringent conditions encode antibodies or antigen binding regions which are competitively inhibited from binding, to a statistically significant degree, by exemplary antibodies with VH and VL as in any one of Ab-1 through Ab-8, as discussed in B, above.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Stringent hybridization conditions embrace: 1) moderate stringency conditions which include hybridization in 40 to 45% formamide, 1 M NaCl, 1% SDS at 37° C., and a wash in 0.5× to 1×SSC at 55 to 60° C.; and, 2) high stringency conditions include hybridization in 50% formamide, 1 M NaCl, 1% SDS at 37° C., and a wash in 0.1×SSC at 60 to 65° C. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y. (1993); and Current Protocols in Molecular Biology, Chapter 2, Ausubel, et al., Eds., Greene Publishing and Wiley-Interscience, New York (1995).

(4) The present invention also includes isolated nucleic acids comprising polynucleotides of the present invention, wherein the polynucleotides have a specified sequence identity at the nucleotide level to a polynucleotide as disclosed in sections (1) and (2), above. Identity can be calculated using, for example, the BLAST, CLUSTALW, or GAP algorithms under default parameters. The percentage of identity to a reference sequence is at least 80% and, rounded upwards to the nearest integer, can be expressed as an integer selected from the group of integers consisting of from 80 to 99. Thus, for example, the percentage of identity to a reference sequence can be at least 85%, 90%, 92%, 94%, 95%, 97%, or 99%, or any integeric value between. Unless otherwise indicated, sequence identity is calculated according to the GAP program (a part of the GCG Wisconsin Package, version 10.3 (Accelrys, San Diego, Calif.)) using its default parameters. Often, the polynucleotides of this embodiment encode antibodies or antigen binding regions which specifically bind to the extracellular domain of human CD148. In some embodiments, the polynucleotides encode antibodies or antigen binding regions which are competitively inhibited from binding, to a statistically significant degree, by exemplary antibodies or antigen binding regions comprising VH and VL chains as in Ab-1 through Ab-8 as discussed in B, above.

GAP (Global Alignment Program) can also be used to compare a polynucleotide or polypeptide of the present invention with a reference sequence. GAP uses the algorithm of Needleman and Wunsch (J. Mol. Biol. 48: 443-453, 1970) to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10.3 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 100. Thus, for example, the gap creation and gap extension penalties can each independently be: 0, 1, 2, 3, 4, 5, 6, 7, 8, 9, 10, 15, 20, 30, 40, 50, 60 or greater. Multiple alignment of the sequences can be performed using the CLUSTAL method of alignment (Higgins and Sharp (1989) CABIOS. 5:151-153) with the default parameters (GAP PENALTY=10, GAP LENGTH PENALTY=10). Default parameters for pairwise alignments using the CLUSTAL method are KTUPLE 1, GAP PENALTY=3, WINDOW=5 and DIAGONALS SAVED=5.

### D. Construction of Antibodies and Antigen Binding Regions

The antibodies and antigen binding regions of the present invention can be constructed by any number of different methods, including, via immunization of animals (e.g., with an antigen that elicits the production of antibodies that specifically bind to and competitively inhibit the binding of at least one of an antibody of Ab-1 through Ab-8); via hybridomas (e.g., employing B-cells from transgenic or non-transgenic animals); via recombinant methods (e.g., CHO transfectomas; see, Morrison, S. (1985) Science 229:1202)), or, in vitro synthetic means (e.g., solid-phase polypeptide synthesis).

In some embodiments, the antibodies and antigen binding regions are human or humanized. Methods for humanizing non-human antibodies are well known in the art. Humanization can be essentially performed following the method of Winter and co-workers (Jones et al., Nature, 321:,522 (1986); Riechmann et al., Nature, 332: 323 (1988); Verhoeyen et al., Science, 239: 1534 (1988)). Briefly, human constant region genes are joined to appropriate human or non-human variable region genes. For example, the amino acid sequences which represent the antigen binding sites (CDRs, or complimentarity determining regions) of a parent murine monoclonal antibody are grafted at the DNA level onto human variable region framework sequences. The sequences of human constant regions genes may be found in Kabat et al. (1991) Sequences of Proteins of Immunological Interest, N.I.H. publication no. 91-3242. Human C region genes are readily available from known clones. The choice of antibody isotype will be guided by the desired effector functions, such as complement fixation, or activity in antibody-dependent cellular cytotoxicity. In certain embodiments, the isotype is IgG₂.

Human or humanized antibodies or antigen binding regions can also be generated through display-type technologies, including, without limitation, phage display, retroviral display, ribosomal display, and other techniques, using techniques well known in the art and the resulting molecules can be subjected to additional maturation, such as affinity maturation, as such techniques are well known in the art. Hanes and Plucthau PNAS USA 94:4937-4942 (1997) (ribosomal display), Parmley and Smith Gene 73:305-318 (1988) (phage display), Scott TIBS 17:241-245 (1992), Cwirla et al. PNAS USA 87:6378-6382 (1990), Russel et al. Nucl. Acids Research 21:1081-1085 (1993), Hoganboom et al. Immunol. Reviews 130:43-68 (1992), Chiswell and McCafferty TIBTECH 10:80-84 (1992), and U.S. Pat. No. 5,733,743.

Identification of suitable human antibody sequences may be facilitated by computer modeling. Modeling is well known in the art, and are used, for example, to avoid unnatural juxtaposition of non-human CDR regions with human variable framework regions, which can result in unnatural conformational restraints and concomitant loss of binding affinity. Computer hardware and software for producing three-dimensional images of immunoglobulin molecules are widely available. In general, molecular models are produced starting from solved structures for immunoglobulin chains or domains thereof. The chains to be modeled are compared for amino acid sequence similarity with chains or domains of solved three dimensional structures, and the chains or domains showing the greatest sequence similarity are selected as starting points for construction of the molecular model. The solved starting structures are modified to allow for differences between the actual amino acids in the immunoglobulin chains or domains being modeled, and those in the starting structure. The modified structures are then assembled into a composite immunoglobulin. Finally, the model is refined by energy minimization and by verifying that all atoms are within appropriate distances from one another and that bond lengths and angles are within chemically acceptable limits.

Transgenic non-human animals (e.g. mice) can be produced that are capable, upon immunization, of producing a full repertoire of human antibodies in the absence of endogenous immunoglobulin production. For example, the homozygous deletion of the antibody heavy-chain joining region (JH) gene in chimeric and germ-line mutant mice results in complete inhibition of endogenous antibody production. Transfer of the human germ-line immunoglobulin gene array in such germline mutant mice will result in the production of human antibodies upon antigen challenge. See, e.g., Jakobovits et al., Proc. Natl. Acad. Sci. USA, 90: 2551 (1993); Jakobovits et al., Nature, 362: 255 (1993); Bruggermann et al., Year in Immunol., 20 7: 33 (1993). Commercially accessible transgenic mice strains such as XenoMouse have been described; see, Green et al. Nature Genetics 7:13-21 (1994).

Recombinant methods for producing antibodies or antigen binding regions of the present invention begin with the isolated nucleic acid of desired regions of the immunoglobulin heavy and light chains such as those present in any of Ab-1 through Ab-8. Such regions can include, for example, all or part of the variable region of the heavy and light chains. Such regions can, in particular, include at least one of the CDRs of the heavy and/or light chains, and often, at least one CDR pair from Ab-1 through Ab-8. A nucleic acid encoding an antibody or antigen binding region of the invention can be directly synthesized by methods of in vitro oligonucleotide synthesis known in the art. Alternatively, smaller fragments can be synthesized and joined to form a larger fragment using recombinant methods known in the art. Antibody binding regions, such as for Fab or F(ab′)₂, may be prepared by cleavage of the intact protein, e.g. by protease or chemical cleavage. Alternatively, a truncated nucleic acid can be designed.

The nucleic acids of the present invention can be constructed by any number of means, such as through recombinant technology, via in vitro synthetic means (e.g., solid phase phosphoramidite synthesis), or combinations thereof. Such methods are well known to those of ordinary skill in the art. See, for example, Current Protocols in Molecular Biology, Ausubel, et al., Eds., Greene Publishing and Wiley-Interscience, New York (1995). The isolated nucleic acids of the present invention can also be prepared by direct chemical synthesis by methods such as the phosphotriester method of Narang et at, Meth. Enzymol. 68: 90-99 (1979); the phosphodiester method of Brown et al, Meth. Enzymol. 68: 109-151 (1979); the diethylphosphoramidite method of Beaucage et al., Tetra. Lett. 22: 1859-1862 (1981); the solid phase phosphoramidite triester method described by Beaucage and Caruthers, Tetra. Letts. 22(20): 1859-1862 (1981), e.g., using an automated synthesizer, e.g., as described in Needham-VanDevanter et al., Nucleic Acids Res., 12: 6159-6168 (1984); and, the solid support method of U.S. Pat. No. 4,458,066.

To express the antibodies or antigen binding regions thereof, DNAs encoding partial or full-length light and heavy chains, can be obtained by standard molecular biology techniques (e.g., PCR amplification, site directed mutagenesis) and can be inserted into expression vectors such that the genes are operatively linked to transcriptional and translational regulatory sequences. Nucleic acids encoding an antibody or antigen binding region of the invention can be cloned into a suitable expression vector and expressed in a suitable host. A suitable vector and host cell system can allow, for example, co-expression and assembly of the variable heavy and variable light chains of at least one of Ab-1 through Ab-8, or CDR containing polypeptides thereof. Suitable systems for expression can be determined by those skilled in the art. In some embodiments, the expression vectors are split DHFR vectors, PDC323 or PDC324; see, McGrew, J. T. and Bianchi, A. A. (2002) “Selection of cells expressing heteromeric proteins”, U.S. patent Application No. 20030082735; and, Bianchi, A. A. and McGrew, J. T. (2003) “High-level expression of full antibodies using trans-complementing expression vectors”. Bioengineering and Biotechnology. 84 (4): 439-444.

Nucleic acids comprising polynucleotides of the present invention can be used in transfection of a suitable mammalian or nonmammalian host cells. In some embodiments, for expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most typical because such eukaryotic cells, and in particular mammalian cells, are more likely than prokaryotic cells to assemble and secrete a properly folded and immunologically active antibody or antigen binding region.

Expression vectors include plasmids, retroviruses, cosmids, YACs, EBV derived episomes, and the like. A convenient vector is one that encodes a functionally complete human CH (constrant heavy) or CL (constant light) immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions.

The expression vector and expression control sequences are chosen to be compatible with the expression host cell used. The antibody variable heavy chain nucleic acid and the antibody variable light chain nucleic acids of the present invention can be inserted into separate vectors or, frequently, both genes are inserted into the same expression vector. The nucleic acids can be inserted into the expression vector by standard methods (e.g., ligation of complementary restriction sites on the antibody nucleic acid fragment and vector, or blunt end ligation if no restriction sites are present). The heavy and light chain variable regions of Ab-1 through Ab-8, described herein, can be used to create full-length antibody genes of any antibody isotype by inserting them into expression vectors already encoding heavy chain constant and light chain constant regions of the desired isotype (and subclass) such that the VH segment is operatively linked to the CH segment(s) within the vector and the VL segment is operatively linked to the CL segment within the vector. Additionally or alternatively, the expression vector can encode a signal peptide that facilitates secretion of the antibody or antigen binding region chain from a host cell. The antibody or antigen binding region chain gene can be cloned into the vector such that the signal peptide is linked in-frame to the amino terminus of the antibody/antigen binding region chain gene. The signal peptide can be an immunoglobulin signal peptide or a heterologous signal peptide (i.e., a signal peptide from a non-immunoglobulin protein).

In addition to the CDR comprising sequence, the expression vectors of the invention carry regulatory sequences that control the expression of the sequence in a host cell. The term “regulatory sequence” is intended to includes promoters, enhancers and other expression control elements (e.g., polyadenylation signals) that control the transcription or translation of the antibody chain genes. Such regulatory sequences are described, for example, in Goeddel; Gene Expression Technology. Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, and the like. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP)) and polyoma. Alternatively, nonviral regulatory sequences may be used, such as the ubiquitin promoter or beta-globin promoter.

In addition to the antibody or antigen binding region nucleic acids and regulatory sequences, the expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

Preferred mammalian host cells for expressing the recombinant antibodies or antigen binding regions of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NS/0 myeloma cells, COS cells and SP2.0 cells. In particular for use with NS/0 myeloma cells, another preferred expression system is the GS gene expression system disclosed in WO 87/04462, WO 89/01036 and EP 338841. When expression vectors of the invention are introduced into mammalian host cells, the antibodies or antigen binding regions are produced by culturing the host cells in the appropriate culture media for a period of time sufficient to allow for expression of the antibody or antigen binding region in the host cells or, more preferably, secretion of the antibody or antigen binding region into the culture medium in which the host cells are grown.

Once expressed, antibodies and antigen binding regions of the invention can be purified for isolation according to standard methods in the art, including HPLC purification, fraction column chromatography, gel electrophoresis and the like (see, e.g., Scopes, Protein Purification, Springer-Verlag, NY, 1982). In certain embodiments, polypeptides are purified using chromatographic and/or electrophoretic techniques. Exemplary purification methods include, but are not limited to, precipitation with ammonium sulphate; precipitation with PEG; immunoprecipitation; heat denaturation followed by centrifugation; chromatography, including, but not limited to, affinity chromatography (e.g., Protein-A-Sepharose), ion exchange chromatography, exclusion chromatography, and reverse phase chromatography; gel filtration; hydroxylapatite chromatography; isoelectric focusing; polyacrylamide gel electrophoresis; and combinations of such and other techniques. In certain embodiments, a polypeptide is purified by fast protein liquid chromatography or by high pressure liquid chromotography (HPLC).

A useful strategy for the PEGylation of synthetic peptides consists of combining, through forming a conjugate linkage in solution, a peptide and a PEG moiety, each bearing a special functionality that is mutually reactive toward the other. The peptides can be easily prepared with conventional solid phase synthesis as known in the art. The peptides are “preactivated” with an appropriate functional group at a specific site. The precursors are purified and fully characterized prior to reacting with the PEG moiety. Ligation of the peptide with PEG usually takes place in aqueous phase and can be easily monitored by reverse phase analytical HPLC. The PEGylated peptides can be easily purified by preparative HPLC and characterized by analytical HPLC, amino acid analysis and laser desorption mass spectrometry.

### E. Compositions and Methods of Treatment and Diagnosis

The present invention provides pharmaceutical compositions comprising antibodies and/or antigen binding regions of the present invention formulated with a pharmaceutically acceptable carrier. In some embodiments, the pharmaceutically acceptable carrier is suitable for administration in human subjects.

As used herein, “pharmaceutically acceptable carrier” includes any and all solvents, dispersion media, coatings, antibacterial and antifungal agents, isotonic and absorption delaying agents, and the like that are physiologically compatible when administered to a particular subject. Pharmaceutical formulations of the present invention include those suitable for, and intended for use in, intravenous, intramuscular, subcutaneous, spinal or epidermal administration (e.g., by injection or infusion), oral, nasal, topical (including buccal and sublingual), rectal, vaginal and/or parenteral administration. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Depending on the route of administration, the active compound (i.e., the antibody or antigen binding region) may be coated in a material to protect the compound from the action of acids and other natural conditions that may inactivate the compound. In another embodiment, the pharmaceutical compositions are formulated with a carrier that is pharmaceutically acceptable in humans. The amount of active ingredient which can be combined with a carrier material to produce a single dosage form will generally be that amount of the composition which produces a therapeutic effect and/or provide a useful diagnostic outcome. Generally, out of one hundred percent, this amount will range from about 0.01% to about 99% of active ingredient, often from about 0.1% to about 70%, or, from about 1% to about 30%. In some embodiments, the compositions of the present invention are admixed with a pharmaceutically acceptable carrier at a concentration of at least 1, 10, 25, 50, 100, 250 micrograms per milliliter, often 1 to 30, or 5 to 30 micrograms per milliliter.

The various therapeutic moieties described herein that improve the therapeutic and/or diagnostic benefit can be covalently linked, directly or indirectly (e.g., via a linking group) to an antibody or antigen binding region to yield a “conjugate”. Any “linking” group is optional. When present, its chemical structure is not critical, since it serves primarily as a spacer. The linker is often made up of amino acids linked together by peptide bonds. One or more of these amino acids may be glycosylated, as is well understood by those in the art. Non-peptide linkers are also possible. An exemplary non-peptide linker is a PEG (polyethylene glycol) linker, and has a molecular weight of 100 to 5000 kDa, often 100 to 500 kDa.

Techniques for conjugating such therapeutic moieties to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies'84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Pharmaceutical compositions of the invention can be administered in combination therapy, i.e., combined with other agents. In some embodiments, the combination therapy can include a composition of the present invention with at least one anti-tumor agent or other conventional therapy. In some embodiments, the combination comprises a composition of the present invention (e.g., an antibody or antigen binding region) in combination with at least one anti-angiogenic agent. Agents are inclusive of, but not limited to, in vitro synthetically prepared chemical compositions, antibodies, antigen binding regions, radionuclides, and combinations and conjugates thereof. An agent can be an agonist, antagonist, allosteric modulator, toxin or, more generally, may act to inhibit or stimulate its target (e.g., receptor or enzyme activation or inhibition), and thereby promote cell death or arrest cell growth.

Exemplary anti-tumor agents include HERCEPTIN™ (trastuzumab), which may be used to treat breast cancer and other forms of cancer, and RITUXAN™ (rituximab), ZEVALIN™ (ibritumomab tiuxetan), and LYMPHOCIDE™ (epratuzumab), which may be used to treat non-Hodgkin's lymphoma and other forms of cancer, GLEEVAC™ which may be used to treat chronic myeloid leukemia and gastrointestinal stromal tumors, and BEXXAR™ (iodine 131 tositumomab) which may be used for treatment of non-Hodgkins's lymphoma.

Exemplary anti-angiogenic agents include ERBITUX™ (IMC-C225), KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF, or soluble VEGF receptors or a ligand binding region thereof) such as AVASTIN™ or VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as ABX-EGF (panitumumab), IRESSA™ (gefitinib), TARCEVA™ (erlotinib), anti-Ang1 and anti-Ang2 agents (e.g., antibodies or antigen binding regions specifically binding thereto or to their receptors, e.g., Tie2/Tek), and anti-Tie-2 kinase inhibitory agents (e.g., antibodies or antigen binding regions that specifically bind thereto). The pharmaceutical compositions of the present invention can also include one or more agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor “c-met”.

Other anti-angiogenic agents include Campath, IL-8, B-FGF, Tek antagonists (Ceretti et al., U.S. Publication No. 2003/0162712; U.S. Pat. No. 6,413,932), anti-TWEAK agents (e.g., specifically binding antibodies or antigen binding regions, or soluble TWEAK receptor antagonists; see, Wiley, U.S. Pat. No. 6,727,225), ADAM distintegrin domain to antagonize the binding of integrin to its ligands (Fanslow et al., U.S. Publication No. 2002/0042368), specifically binding anti-eph receptor and/or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto).

Additional anti-angiogenic/anti-tumor agents include: SD-7784 (Pfizer, USA); cilengitide (Merck KGaA, Germany, EPO 770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol, (EntreMed, USA); TLC ELL-12, (Elan, Ireland); anecortave acetate, (Alcon, USA); alpha-D148 Mab, (Amgen, USA); CEP-7055, (Cephalon, USA); anti-Vn Mab, (Crucell, Netherlands) DAC:antiangiogenic, (ConjuChem, Canada); Angiocidin, (InKine Pharmaceutical, USA); KM-2550, (Kyowa Hakko, Japan); SU-0879, (Pfizer, USA); CGP-79787, (Novartis, Switzerland, EP 970070); ARGENT technology, (Ariad, USA); YIGSR-Stealth, (Johnson & Johnson, USA); fibrinogen-E fragment, (BioActa, UK); angiogenesis inhibitor, (Trigen, UK); TBC-1635, (Encysive Pharmaceuticals, USA); SC-236, (Pfizer, USA); ABT-567, (Abbott, USA); Metastatin, (EntreMed, USA); angiogenesis inhibitor, (Tripep, Sweden); maspin, (Sosei, Japan); 2-methoxyestradiol, (Oncology Sciences Corporation, USA); ER-68203-00, (IVAX, USA); Benefin, (Lane Labs, USA); Tz-93, (Tsumura, Japan); TAN-1120, (Takeda, Japan); FR-111142, (Fujisawa, Japan, JP 02233610); platelet factor 4, (RepliGen, USA, EP 407122); vascular endothelial growth factor antagonist, (Borean, Denmark); cancer therapy, (University of South Carolina, USA); bevacizumab (pINN), (Genentech, USA); angiogenesis inhibitors, (SUGEN, USA); XL 784, (Exelixis, USA); XL 647, (Exelixis, USA); MAb, alpha5beta3 integrin, second generation, (Applied Molecular Evolution, USA and MedImmune, USA); gene therapy, retinopathy, (Oxford BioMedica, UK); enzastaurin hydrochloride (USAN), (Lilly, USA); CEP 7055, (Cephalon, USA and Sanofi-Synthelabo, France); BC 1, (Genoa Institute of Cancer Research, Italy); angiogenesis inhibitor, (Alchemia, Australia); VEGF antagonist, (Regeneron, USA); rBPI 21 and BPI-derived antiangiogenic, (XOMA, USA); PI 88, (Progen, Australia); cilengitide (pINN), (Merck KGaA, German; Munich Technical University, Germany, Scripps Clinic and Research Foundation, USA); cetuximab (INN), (Aventis, France); AVE 8062, (Ajinomoto, Japan); AS 1404, (Cancer Research Laboratory, New Zealand); SG 292, (Telios, USA); Endostatin, (Boston Childrens Hospital, USA); ATN 161, (Attenuon, USA); ANGIOSTATIN, (Boston Childrens Hospital, USA); 2-methoxyestradiol, (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aetema, Canada); vaccine, angiogenesis, (EntreMed, USA); urokinase plasminogen activator inhibitor, (Dendreon, USA); oglufanide (pINN), (Melmotte, USA); HIF-1alfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol, (EntreMed, USA); anginex, (Maastricht University, Netherlands, and Minnesota University, USA); ABT 510, (Abbott, USA); AAL 993, (Novartis, Switzerland); VEGI, (ProteomTech, USA); tumor necrosis factor-alpha inhibitors, (National Institute on Aging, USA); SU 11248, (Pfizer, USA and SUGEN USA); ABT 518, (Abbott, USA); YH16, (Yantai Rongchang, China); S-3APG, (Boston Childrens Hospital, USA and EntreMed, USA); MAb, KDR, (ImClone Systems, USA); MAb, alpha5 beta1, (Protein Design, USA); KDR kinase inhibitor, (Celltech Group, UK, and Johnson & Johnson, USA); GFB 116, (South Florida University, USA and Yale University, USA); CS 706, (Sankyo, Japan); combretastatin A4 prodrug, (Arizona State University, USA); chondroitinase AC, (IBEX, Canada); BAY RES 2690, (Bayer, Germany); AGM 1470, (Harvard University, USA, Takeda, Japan, and TAP, USA); AG 13925, (Agouron, USA); Tetrathiomolybdate, (University of Michigan, USA); GCS 100, (Wayne State University, USA) CV 247, (Ivy Medical, UK); CKD 732, (Chong Kun Dang, South Korea); MAb, vascular endothelium growth factor, (Xenova, UK); irsogladine (INN), (Nippon Shinyaku, Japan); RG 13577, (Aventis, France); WX 360, (Wilex, Germany); squalamine (pINN), (Genaera, USA); RPI 4610, (Sirna, USA); cancer therapy, (Marinova, Australia); heparanase inhibitors, (InSight, Israel); KL 3106, (Kolon, South Korea); Honokiol, (Emory University, USA); ZK CDK, (Schering AG, Germany); ZK Angio, (Schering AG, Germany); ZK 229561, (Novartis, Switzerland, and Schering AG, Germany); XMP 300, (XOMA, USA); VGA 1102, (Taisho, Japan); VEGF receptor modulators, (Pharmacopeia, USA); VE-cadherin-2 antagonists, (ImClone Systems, USA); Vasostatin, (National Institutes of Health, USA);vaccine, Flk-1, (ImClone Systems, USA); TZ 93, (Tsumura, Japan); TumStatin, (Beth Israel Hospital, USA); truncated soluble FLT 1 (vascular endothelial growth factor receptor 1), (Merck & Co, USA); Tie-2 ligands, (Regeneron, USA); and, thrombospondin 1 inhibitor, (Allegheny Health, Education and Research Foundation, USA).

The compositions of the present invention can be coupled to radionuclides, such as 1311, 90Y, 105Rh, indium-111, etc., as described in Goldenberg, D. M. et al. (1981) Cancer Res. 41: 4354-4360, and in EP 0365 997. In another aspect the invention relates to an immunoconjugate comprising an antibody according to the invention linked to a radioisotope, cytotoxic agent (e.g., calicheamicin and duocarmycin), a cytostatic agent, or a chemotherapeutic agent selected from the group consisting of nitrogen mustards (e.g., cyclophosphamide and ifosfamide), aziridines (e.g., thiotepa), alkyl sulfonates (e.g., busulfan), nitrosoureas (e.g., carmustine and streptozocin), platinum complexes (e.g., carboplatin and cisplatin), non-classical alkylating agents (e.g., dacarbazine and temozolamide), folate analogs (e.g., methotrexate), purine analogs (e.g., fludarabine and mercaptopurine), adenosine analogs (e.g., cladribine and pentostatin), pyrimidine analogs (e.g., fluorouracil (alone or in combination with leucovorin) and gemcitabine), substituted ureas (e.g., hydroxyurea), antitumor antibiotics (e.g., bleomycin and doxorubicin), epipodophyllotoxins (e.g., etoposide and teniposide), microtubule agents (e.g., docetaxel and paclitaxel), camptothecin analogs (e.g., irinotecan and topotecan), enzymes (e.g., asparaginase), cytokines (e.g., interleukin-2 and interferon-.alpha, monoclonal antibodies (e.g., trastuzumab and bevacizumab), recombinant toxins and immunotoxins (e.g., recombinant cholera toxin-B and TP-38), cancer gene therapies, physical therapies (e.g., hyperthermia, radiation therapy, and surgery) and cancer vaccines (e.g., vaccine against telomerase). Co-administration of the human anti-CD148 antibodies, or antigen binding fragments thereof, of the present invention with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells which would render them unreactive with the antibody.

In another aspect the pharmaceutical composition comprises one or more further anti-inflammatory agents selected from the group consisting of aspirin and other salicylates, steroidal drugs, NSAIDs (nonsteroidal anti-inflammatory drugs) (e.g., ibuprofen, fenoprofen, naproxen, sulindac, diclofenac, piroxicam, ketoprofen, diflunisal, nabumetone, etodolac, oxaprozin, and indomethacin), Cox-2 inhibitors (e.g., rofecoxib and celecoxib), and DMARDs (disease modifying antirheumatic drugs) (e.g., methotrexate, hydroxychloroquine, sulfasalazine, azathioprine, pyrimidine synthesis inhibitors (e.g., leflunomide), IL-1 receptor blocking agents (e.g., anakinra), TNF-.alpha. blocking agents (e.g., etanercept, infliximab and adalimumab), anti-IL-6R antibodies, CTLA4Ig, and anti-IL-15 antibodies).

In another aspect the pharmaceutical composition comprises one or more further anti-psoriasis agents selected from the group consisting of coal tar, A vitamin, anthralin, calcipotrien, tarazotene, corticosteroids, methotrexate, retinoids (e.g., acitretin), cyclosporine, etanercept, alefacept, efaluzimab, 6-thioguanine, mycophenolate mofetil, tacrolimus (FK-506), and hydroxyurea.

The active compounds of the pharmaceutical compositions can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978. To administer a compound of the invention by certain routes of administration, it may be

necessary to coat the compound with, or co-administer the compound with, a material to prevent its inactivation. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. For example, the compound may be administered to a subject in an appropriate vehicle, for example, liposomes. Liposomes include water-in-oil-in-water CGF emulsions as well as conventional liposomes (Strejan et al. (1984) J. Neuroimmunol. 7:27).

Pharmaceutical compositions typically must be sterile and stable under the conditions of manufacture and storage. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin. Sterile injectable solutions can be prepared by incorporating the active compound(s) in the required amount in an appropriate solvent with one or a combination of ingredients enumerated above, as required, followed by sterilization microfiltration. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

An antibody or antigen binding region of the invention may be administered, for example, once or more than once, e.g., at regular intervals over a period of time. In particular embodiments, the antibody is administered over a period of at least a month or more, e.g., for one, two, or three months or even indefinitely. For treating chronic conditions, long-term treatment is generally most effective. However, for treating acute conditions, administration for shorter periods, e.g. from one to six weeks, may be sufficient. In general, the antibody is administered until the patient manifests a medically relevant degree of improvement over baseline for the chosen indicator or indicators. A “therapeutically effective amount” is an amount of the pharmaceutical composition of the present invention that when administered to a subject ameliorates or prevents a given condition to a statistically significant degree.

Without being bound by theory, it is believed that the pharmaceutical compositions of the present invention inhibit tumor growth by inhibiting the growth of blood vessels supplying nutrients to the tumor. In the treatment of tumor angiogenesis, a therapeutically effective amount is, in some embodiments, sufficient to inhibit angiogenesis and/or tumor growth by at least about 10%, 20%, 30%, 40%, 50%, or 60%, relative to untreated subjects. The ability of a compound to inhibit angiogenesis and tumor growth can be evaluated in an animal model system predictive of efficacy in human tumors. Alternatively, this property of a composition can be evaluated by examining the ability of the compound to inhibit, such inhibition in vitro by assays known to the skilled practitioner (e.g., corneal pocket or planar migration assays).

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. One of ordinary skill in the art would be able to determine administered amounts based on such factors as the subject's size, the severity of the subject's symptoms, and the particular composition or route of administration selected. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. Particular embodiments of the present invention involve administering an antibody at a dosage of from about 1 ng of antibody per kg of subject's weight per day (“1 ng/kg/day”) to about 10 mg/kg/day, more typically from about 500 ng/kg/day to about 5 mg/kg/day, and often from about 5 μg/kg/day to about 2 mg/kg/day, to a subject.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. In general, a suitable daily dose of a compositions of the invention will be that amount of the compound which is the lowest dose effective to produce a therapeutic effect. Such an effective dose will generally depend upon the factors described above. It is preferred that administration be intravenous, intramuscular, intraperitoneal, or subcutaneous, preferably administered proximal to the site of the target. If desired, the effective daily dose of a therapeutic compositions may be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation (composition).

Therapeutic compositions can be administered with medical devices known in the art. For example, in a preferred embodiment, a therapeutic composition of the invention can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

In certain embodiments, the antibodies or antigen binding regions of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134), different species of which may comprise the formulations of the inventions, as well as components of the invented molecules; p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

In terms of compositions, kits and/or medicaments of the invention, the combined effective amounts of the therapeutic agents may be comprised within a single container or container means, or comprised within distinct containers or container means. The cocktails will generally be admixed together for combined use. Agents formulated for intravenous administration will often be preferred. Imaging components may also be included. The kits may also comprise written or web-accessible instructions for using the at least a first antibody and the one or more other biological agents included.

### F. Uses and Methods of the Invention

The compositions of the present invention (e.g., pharmaceutical compositions, and antibody or antigen binding region and conjugates thereof) have in vitro and in vivo diagnostic and therapeutic utilities. For example, these molecules can be administered to cells in culture, e.g., in vitro or ex vivo, or in a subject, e.g., in vivo, to treat or diagnose a variety of disorders. As used herein, the term “subject” is intended to include human and non-human mammals. A therapeutically effective amount of a pharmaceutical composition of the invention is administered to a mammalian subject, typically a human patient. The amount administered is sufficient to ameliorate or prevent a condition (e.g., tumor growth, angiogenesis, or inflammation) to a statistically significant extent. Treatment, in this fashion, encompasses alleviation or prevention of at least one symptom of a disorder, or reduction of disease severity, and the like. The therapeutic methods of the invention need not effect a complete “cure”, or eradicate every symptom or manifestation of a disease, to constitute a viable therapeutic method. As is recognized in the pertinent field, the therapeutic methods may reduce the severity of a given disease state, but need not abolish every manifestation of the disease to be regarded as useful therapeutic methods.

Compositions of the present invention can be used in therapeutic applications to inhibit angiogenesis. It is well known to those of ordinary skill in the art that as aberrant angiogenesis occurs in a wide range of diseases and disorders, a given anti-angiogenic therapy, once shown to be effective in any acceptable model system, can be used to treat the entire range of diseases and disorders connected with angiogenesis. The methods and uses of the present invention are particularly intended for use in mammals, particularly human patients that have, or are at risk for developing, any form of vascularized tumor including, for example, bladder, breast, kidney, ovarian, prostate, renal cell, squamous cell, lung (non-small cell), uterine/cervical, pancreatic, colorectal, stomach, ovarian, prostate squamous cell, lung (non-small cell), esophageal, and head and neck cancer. Exemplary cancers include, but are not limited to, breast cancer, colorectal cancer, gastric carcinoma, glioma, head and neck squamous cell carcinoma, hereditary and sporadic papillary renal carcinoma, leukemia, lymphoma, Li-Fraumeni syndrome, malignant pleural mesothelioma, melanoma, multiple myeloma, non-small cell lung carcinoma, osteosarcoma, ovarian cancer, pancreatic cancer, prostate cancer, small cell lung cancer, synovial sarcoma, thyroid carcinoma, and transitional cell carcinoma of urinary bladder.

The methods and uses of the invention are further intended for the treatment of mammals, in particular, human patients that have, or are at risk for developing, macular degeneration, including age-related macular degeneration; arthritis, including rheumatoid arthritis; atherosclerosis and atherosclerotic plaques; diabetic retinopathy and other retinopathies; thyroid hyperplasias, including Grave's disease; hemangioma; neovascular glaucoma; and psoriasis, arterioyenous malformations (AVM), meningioma, and vascular restenosis, including restenosis following angioplasty. Other intended targets of the therapeutic methods and uses are animals and patients that have, or are at risk for developing, angiofibroma, dermatitis, endometriosis, hemophilic joints, hypertrophic scars, inflammatory diseases and disorders, pyogenic granuloma, scleroderma, synovitis, trachoma and vascular adhesions. The pharmaceutical compositions of the invention also are useful for in vivo imaging, wherein an antibody labeled with a detectable moiety such as a radio-opaque agent or radioisotope is administered to a subject, preferably into the bloodstream, and the presence and location of the labeled antibody in the subject is assayed. This imaging technique is useful in the staging and treatment of neoplasms or bone disorders. The antibody may be labeled with any moiety that is detectable in a host, whether by nuclear magnetic resonance, radiology, or other detection means known in the art. In preferred embodiments the subject is human.

In vitro binding assays are also provided by the compositions of the invention. Immunological binding assays typically utilize a capture agent to bind specifically to and often immobilize the analyte target antigen. The capture agent is a moiety that specifically binds to the analyte. In one embodiment of the present invention, the capture agent is an antibody or antigen binding region thereof that specifically binds the extracellular domain of human CD148. These immunological binding assays are well known in the art (Asai, ed., Methods in Cell Biology, Vol. 37, Antibodies in Cell Biology, Academic Press, Inc., New York (1993)).

### EXAMPLES

The following examples, including the experiments conducted and results achieved, are provided for illustrative purposes only and are not to be construed as limiting the present invention.

### Example 1

Example 1 is a description of the screening procedure used to identify parental versions of the antibody heavy and light variable chains of the present invention.

Recombinant scFv (single chain variable fragment) phage display libraries from Cambridge Antibody Technologies (CAT, Cambridge UK) were interrogated in vitro using against huCD148 protein targets. We then screened over 10,000 clones recovered by phage display (2^{nd} and 3^{rd} round outputs) and identified >250 unique scFv antibodies that specifically bind huCD148 and consolidated 83 of these reagents for further study, based upon their predicted therapeutic potential. For example, several antibodies were isolated that appear to compete for binding to the same epitope as that of another anti-huCD148 antibody (Ab-X) as measured by competitive ELISA or TRF (time-resolved fluorescence) report. Other antibodies were consolidated based upon their relatively high signal:noise ratio as indicated by ELISA or TRF (e.g., target binding compared to streptavidin binding) or because they cross-reacted to the murine CD148 ortholog. All anti-CD148 antibodies were tested for their ability to cross-react with muCD148 (for in vivo mouse studies) and to bind cell-expressed huCD148. We expressed and confirmed binding activity and specificity of 8 scFv human Ab-X analog antibodies exhibiting the relatively highest degree of competition with Ab-X as either IgG4s, maxibodies (bivalent scFv-Fcs) or both, as well as 7 other clones. We cloned the VH and VL genes for the original mouse Ab-X antibody, subcloned these genes into various antibody “platforms” to serve as positive controls in comparative binding (i.e., ELISA or FACS) and functional in vitro and in vivo assays. Primary functional screening of our initial “top 14” leading candidate antibodies is now complete. Eight of these clones agonize huCD148 in an in vitro planar migration assay. Moreover, four of these lead clones that cross-react to muCD148 inhibit FGF-2 induced angiogenesis in our in vivo mouse corneal pocket assay. The eight antibodies Ab-1 through Ab-8 were capable of inhibiting human endothelial cell migration and/or inhibited angiogenesis in the corneal angiogenesis assay with at least 20% capability as compared with controls in vitro at a concentration of 20 μg/ml.

What is claimed is:

1. An isolated antibody or an antigen binding region thereof, comprising a polypeptide sequence having at least 90% sequence identity to a variable chain sequence selected from the group consisting of: SEQ ID NOS: 2, 4, 6, 8, 10, 12, 14, 16, 18, 20, 22, 24, 26, 28, 30 and 32, and wherein said antibody or antigen binding region specifically binds to the extracellular domain of human CD148.

2. The isolated antibody or an antigen binding region of claim 1, which is competitively inhibited from specifically binding to human CD148 by antibodies having a variable heavy chain and a variable light chains of:
a) SEQ ID NO₂: and SEQ ID NO:4;
b) SEQ ID NO:6 and SEQ ID NO8;
c) SEQ ID NO: 10 and SEQ ID NO: 12;
d) SEQ ID NO: 14 and SEQ ID NO: 16;
e) SEQ ID NO: 18 and SEQ ID NO:20:
f) SEQ ID NO:22 and SEQ ID NO:24;
g) SEQ ID NO:26 and SEQ ID NO:28; or
h) SEQ ID NO:30 and SEQ ID NO:32.

3. The isolated antibody or an antigen binding region of claim 1, comprising a heavy chain variable polypeptide sequence and a light chain variable polypeptide sequence, said sequences having at least 90% sequence identity to the heavy and light chains from at least one of:
i) for Ab-1: SEQ ID NO₂: and SEQ ID NO:4;
j) for Ab-2: SEQ ID NO:6 and SEQ ID NO 8:;
k) for Ab-3: SEQ ID NO: 10 and SEQ ID NO: 12;
l) for Ab-4: SEQ ID NO: 14 and SEQ ID NO: 16;
m) for Ab-5: SEQ ID NO: 18 and SEQ ID NO:20:
n) for Ab-6: SEQ ID NO:22 and SEQ ID NO:24;
o) for Ab-7: SEQ ID NO:26 and SEQ ID NO:28; or
p) for Ab-8: SEQ ID NO:30 and SEQ ID NO:32.

4. The isolated antibody or antigen binding region of claim 3, wherein said sequence identity is 100%.

5. The isolated antibody or antigen binding region of claim 4 wherein said antibody is a human IgG₂.

6. The isolated antibody or antigen binding region of claim 3, wherein said antigen binding region is selected from the group consisting of: Fab, F(ab′)₂, Fv, and, scFv.

7. The isolated antibody or antigen binding region of claim 6, wherein at least one of said Fv or scFV is covalently bound to a human Fc fragment or a constant heavy domain thereof.

8. The antibody or isolated antigen binding region of claim 3 which are competitively inhibited from specifically binding by antibodies having a variable heavy chain and a variable light chains of:
q) SEQ ID NO₂: and SEQ ID NO:4;
r) SEQ ID NO:6 and SEQ ID N08:;
s) SEQ ID NO: 10 and SEQ ID NO: 12;
t) SEQ ID NO:14 and SEQ ID NO:16;
u) SEQ ID NO: 18 and SEQ ID NO:20:
v) SEQ ID NO:22 and SEQ ID NO:24;
w) SEQ ID NO:26 and SEQ ID NO:28; or
x) SEQ ID NO:30 and SEQ ID NO:32.

9. The isolated antibody or antigen binding region of claim 3, wherein binding yields at least 10% inhibition in an HRMEC human renal microvascular endothelial cell planar migration assay.

10. The isolated antibody or antigen binding region of claim 5, covalently bonded to a conjugate.

11. The isolated antibody or isolated antigen binding region of claim 9 which is human or humanized.

12. The isolated antibody or antigen binding region of claim 5, in a carrier pharmaceutically acceptable for administration in humans.

13. The isolated antibody or isolated antigen binding region of claim 12, wherein said antibody or antigen binding region or combination thereof is admixed with said carrier at a concentration of at least around 1 microgram per milliliter.

14. A kit comprising the isolated antibody or isolated antigen binding region of claim 13, wherein said antibody or antigen binding region in is carrier is sealed within a sterile container and wherein said kit further comprises a package insert providing written instructions on dosage of said antibody or antigen binding region for a human patient.

15. An isolated antibody or isolated antigen binding region thereof comprising at least one complementarity determining regions (CDRs), wherein said CDRs are localized at and inclusive of residues:
a) 31-35, 50-66, or, 99-109 of SEQ ID NO: [2];
b) 23-36, 52-58, or, 91-101 of SEQ ID NO: [4]
c) 31-35, 56-66, or, 99-111 of SEQ ID NO: [6]
d) 24-34, 50-56, or, 89-97 of SEQ ID NO: [8];
e) 31-35, 50-66, or, 99-107 of SEQ ID NO: [10];
f) 24-34, 50-66, or, 89-97 of SEQ ID NO: [1,2];
g) 31-35, 50-66, or, 99-112 of SEQ ID NO: [1,4];
h) 23-33, 49-55, or, 88-98 of SEQ ID NO: [1,6];
i) 31-35, 50-66, or, 99-114 of SEQ ID NO: [1,8];
j) 23-33, 49-55, or, 88-98 of SEQ ID NO: [20];
k) 31-35, 50-66, or, 99-107 of SEQ ID NO: [22];
l) 23-35, 51-57, or, 90-101 of SEQ ID NO: [24];
m) 31-35, 50-66, or, 99-107 of SEQ ID NO: [26];
n) 23-35, 51-57, or, 90-100 of SEQ ID NO: [28];
o) 31-35, 50-66, or, 99-112 of SEQ ID NO: [30]; or,
p) 23-35, 51-57, or, 90-100 of SEQ ID NO: [32]; and,
wherein said isolated antibody or antigen binding region specifically binds to the extracellular domain of human CD148.

16. The isolated antibody or isolated antigen binding region of claim 15, comprising the heavy and light chain of said CDR, wherein the heavy and light chain polypeptide residues are selected from the group consisting of:
a) from Ab-1:
i) CDR1: 31-35 of SEQ ID NO: 2 and 23-36 of SEQ ID NO:4
ii) CDR2: 50-66 of SEQ ID NO: 2 and 52-58 of SEQ ID NO:4;
iii) CDR3: 99-109 of SEQ ID NO: 2 and 91-101 of SEQ ID NO:4;
b) from Ab-2:
i) CDR1: 31-35 of SEQ ID NO: 6 and 24-34 of SEQ ID NO:8
ii) CDR2: 56-66 of SEQ ID NO: 6 and 52-58 of SEQ ID NO:8;
iii) CDR3: 99-111 of SEQ ID NO: 6 and 89-97 of SEQ ID NO:8;
c) from Ab-3:
i) CDR1: 31-35 of SEQ ID NO: 10 and 24-34 of SEQ ID NO: 12;
ii) CDR2: 50-66 of SEQ ID NO: 10 and 50-66 of SEQ ID NO: 12;
iii) CDR3: 99-107 of SEQ ID NO: 10 and 89-107 of SEQ ID NO: 12;
d) from Ab-4:
i) CDR1: 31-35 of SEQ ID NO: 14 and 23-33 of SEQ ID NO: 16
ii) CDR2: 50-66 of SEQ ID NO: 14 and 50-66 of SEQ ID NO: 16;
iii) CDR3: 99-112 of SEQ ID NO: 14 and 88-98 of SEQ ID NO: 16;
e) from Ab-5:
i) CDR1: 31-35 of SEQ ID NO: 18 and 23-33 of SEQ ID NO:20
ii) CDR2: 50-66 of SEQ ID NO: 18 and 49-55 of SEQ ID NO:20;
iii) CDR3: 99-114 of SEQ ID NO: 18 and 88-98 of SEQ ID NO:20;
f) from Ab-6:
i) CDR1: 31-35 of SEQ ID NO: 22 and 23-35 of SEQ ID NO:24
ii) CDR2: 50-66 of SEQ ID NO: 22 and 51-57 of SEQ ID NO:24;
iii) CDR3: 99-107 of SEQ ID NO: 22 and 90-101 of SEQ ID NO:24;
g) from Ab-7:
i) CDR1: 31-35 of SEQ ID NO: 26 and 23-35 of SEQ ID NO:28
ii) CDR2: 50-66 of SEQ ID NO: 26 and 51-57 of SEQ ID NO:28;
iii) CDR3: 99-107 of SEQ ID NO: 26 and 90-100 of SEQ ID NO:28; and,
h) from Ab-8:
i) CDR1: 31-35 of SEQ ID NO: 30 and 23-35 of SEQ ID NO:32
ii) CDR2: 50-66 of SEQ ID NO: 30 and 51-57 of SEQ ID NO:32;
iii) CDR3: 99-112 of SEQ ID NO: 30 and 90-100 of SEQ ID NO:32.

17. The isolated antibody or isolated antigen binding region of claim 16, comprising at least two CDR pairs.

18. An isolated nucleic acid comprising a polynucleotide encoding the isolated antibody or isolated antigen binding region of claim 4.

19. An isolated nucleic acid comprising a polynucleotide encoding the isolated antibody or isolated antigen binding region of claim 5.

20. An expression vector comprising the isolated nucleic acid of claim 19.

21. A host cell comprising the expression vector of claim 20.

22. A method of making the isolated antibody or antigen binding region thereof, comprising culturing in culture media the host cell of claim 21 under conditions that permit expression of said antibody or antigen binding region from said expression vector.

23. The method of claim 22, further comprising isolating said antibody or antigen binding region from said culture media.

24. The host cell of claim 22 which is a hybridoma or a transfected cell.

25. The host cell of claim 24, wherein said transfected cell is a CHO cell.

26. The method of claim 23, further comprising conjugating said isolated antibody or antigen binding region to a detectable label, a cytotoxic agent, a lipid, polyethylene glycol, or a carbohydrate.

27. A method of inhibiting, in a human, angiogenesis of angiogenically active vascular endothelial cells expressing a CD148 receptor, comprising administering to said human a therapeutically effective amount of said antibody or said antigenic binding region of claim 13 and inhibiting angiogenesis.

28. The method of claim 27, wherein said angiogenically active vascular endothelial cells form a blood vessel that provides a blood supply to a solid tumor.

29. The method of claim 27, wherein said angiogenically active vascular endothelial cells form a blood vessel that provides a blood supply to inflamed tissue.

30. The method of claim 27, wherein the pharmaceutically acceptable carrier further comprises a second anti-angiogenic agent.

31. A method of inhibiting growth of a cell expressing human CD148, comprising contacting the cell with a therapeutically effective amount of the antibody or antigen binding region of claim 12 such that the growth of the cell expressing human CD148 is inhibited.
